(19) United States
(12) Patent Application Publication
(10) Pub. No.: US 2005/0250738 A1
(43) Pub. Date: Nov. 10, 2005

(54) Taste-masked formulations containing sertraline and sulfoalkyl ether cyclodextrin

(76) Inventors: Gerold L. Mosher, Kansas City, MO (US); Atef A. Gayed, Overland Park, KS (US); Rebecca L. Wedel, Lawrence, KS (US)

Correspondence Address: INNOVAR, LLC, P O BOX 250647, PLANO, TX, 75025, US

(21) Appl. No.: 11/109,303

(22) Filed: Apr. 19, 2005

Related U.S. Application Data
(60) Provisional application No. 60/568,628, filed on May 6, 2004.

(51) Int. Cl. A61K 31/724; A61K 31/137

(52) U.S. Cl. 514/58; 514/649

(57) ABSTRACT

The present invention provides aqueous oral formulations containing sertraline, or a pharmaceutically acceptable salt thereof, and a sulfoalkyl ether cyclodextrin. The liquid formulations are pleasant tasting, convenient to use, and chemically and physically stable. The liquid formulations can be administered directly or diluted before administration. Unlike the commercially available ZOLOFT™ formulation, the liquid formulations herein do not precipitate upon dilution with water, fruit juices, sodas or other pharmaceutically acceptable oral liquid carriers. The sulfoalkyl ether cyclodextrin-containing formulation provides significant advantages over the marketed non-aqueous formulation and other cyclodextrin-containing formulations of sertraline. The formulation can be self-preserved against microbial growth. The SAE-CD-containing formulation of sertraline can be provided in liquid form or as a reconstitutable powder. Both ready-to-use and concentrated liquid formulations can be prepared. The formulation is available as a clear solution or a suspension.

### CROSS-REFERENCE TO RELATED APPLICATIONS

The present claims the benefit of priority of U.S. Provisional Application for patent Ser. No. 60/568,628 filed May 6, 2004, the entire disclosure of which is hereby incorporated by reference.

### FIELD OF THE INVENTION

The present invention relates to improved antidepressant formulations and in particular to a taste-masked oral solution formulation containing sertraline and a sulfoalkyl ether cyclodextrin and to the use thereof in the treatment of antidepressant responsive disorders and diseases.

### BACKGROUND OF THE INVENTION

Sertraline ((1S-cis)-4-(3,4-dichlorophenyl)-1,2,3,4-tetrahydro-N-methyl-1-naphthalenamine) hydrochloride (HCl) is a selective serotonin reuptake inhibitor (SSRI) that is chemically unrelated to other SSRIs, tricyclic, tetracyclic, or other available antidepressant agents. Relative to first generation antidepressants such as monoamine oxidase (MAO) inhibitors or tricyclics, which affect both norepinephrine and dopamine levels, SSRIs possess a milder adverse events profile due to their selectivity for the serotonergic system, rendering them attractive treatment options for adults as well as pediatric populations.

Sertraline HCl is currently marketed in the United States under the trade name ZOLOFT®. It is supplied as 25-, 50- and 100-mg film-coated tablets and as an oral concentrate (20 mg/mL) in multi-dose 60-mL bottles. Due to the limited solubility and bitter taste of sertraline HCl in water, ZOLOFT® Oral Concentrate is supplied as a non-aqueous solution concentrate in 60 mL multi-dose containers. Each milliliter of the formulation contains sertraline hydrochloride equivalent to 20 mg sertraline, glycerin, alcohol (12%), menthol and butylated hydroxytoluene (BHT). Of the five SSRIs on the U.S. market with liquid dosage forms, only ZOLOFT® Oral Concentrate must be diluted prior to administration, due to the bitter taste of the formulation. As per the package insert, patients are instructed to mix the dose with only water, gingerale, lemon/lime soda, lemonade or orange juice and take the dose immediately. Unfortunately, precipitation of the sertraline is often observed upon dilution of the ZOLOF™ formulation with most of those beverages. Due to its alcohol content, ZOLOFT Oral Concentrate is contraindicated with disulfiram (ANTABUSE®).

Sertraline HCl is indicated in the U.S. for social anxiety disorder, major depressive disorder, panic disorder, obsessive-compulsive disorder (OCD), premenstrual dysphoric disorder (PMDD) and post-traumatic stress disorder (PTSD) in adults and OCD in children (ages 6-12) and adolescents (ages 12-17).

Sertraline and some pharmaceutically acceptable acid addition salts thereof, such as the hydrochloride salt, are disclosed in U.S. Pat. No. 4,536,518, (the '518 patent), the disclosure of which is hereby incorporated by reference in its entirety.

Sertraline is useful in the treatment of a wide range of diseases and disorders. The '518 patent discloses that sertraline and derivatives thereof are useful as antidepressant agents. U.S. Pat. No. 5,130,338 discloses the use of sertraline in the treatment of chemical dependencies, including dependencies on alcohol, tobacco and cocaine. U.S. Pat. No. 4,962,128 discloses the use of sertraline in the treatment of anxiety related disorders such as panic disorder, obsessive-compulsive disorder, generalized anxiety disorder, phobias, post traumatic stress disorder and avoidant personality disorder. U.S. Pat. No. 4,940,731 discloses the use of sertraline in the treatment of premature ejaculation. International PCT Publication No. WO 96/22085 discloses the use of sertraline in the treatment of cancer. U.S. Pat. No. 6,245,782 discloses the use of sertraline in the treatment of post myocardial infarction.

Sertraline can be used in combination with other agents for the treatment of a range of diseases and disorders. U.S. Pat. No. 5,597,826 discloses compositions containing a serotonin selective reuptake inhibitor (SSRI), such as sertraline, and an agonist or antagonist of the serotonin 1 (5-HT.sub.1) receptor and the use of such compositions for treating or preventing a condition selected from mood disorders, including depression, seasonal affective disorders and dysthmia, anxiety disorders, including generalized anxiety disorder and panic disorder; agoraphobia, avoidant personality disorder; social phobia; obsessive compulsive disorder; post-traumatic stress disorder; memory disorders including dementia, amnestic disorders and age-associated memory impairment; disorders of eating behavior, including anorexia nervosa and bulimia nervosa; obesity; cluster headache; migraine; pain; Alzheimer's disease; chronic paroxysmal hemicrania; headache associated with vascular disorders; Parkinson's disease, including dementia in Parkinson's disease, neuroleptic-induced parkinsonism and tardive dyskinesias; endocrine disorders such as hyperprolactinaemia; vasospasm (particularly in the cerebral vasculature); hypertension; disorders in the gastrointestinal tract where changes in motility and secretion are involved; sexual dysfunction, including premature ejaculation; and chemical dependencies.

The '518 patent discloses that sertraline and related compounds can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. According to the '518 patent, when aqueous suspensions and/or elixirs are desired for oral administration, the essential active ingredient therein may be combined with various sweetening, or flavoring agents, coloring matter or dyes and, if so desired, emulsifying and/or suspending agents as well, together with such diluents as water, ethanol, propylene glycol, glycerin and various like combinations thereof. The inclusion of cyclodextrins in formulations containing sertraline is not disclosed.

Also according to the '518 patent, when parenteral administration is desired, solutions of the compounds of the invention can be prepared in sesame or peanut oil or in aqueous propylene glycol or N,N-dimethylformamide, as well as sterile aqueous solution of the water soluble, non-toxic mineral and organic acid addition salts, such solutions being suitably buffered if needed, and made isotonic.

Development of an oral liquid dosage form of sertraline is complicated by the objectionable taste and stringency sensation imparted by the drug in liquid form. Oral liquid solutions or suspensions of sertraline such as described in the '518 patent have an objectionable taste.

U.S. Pat. No. 6,727,283, (hereafter referred to as the '283 patent), discloses an essentially nonaqueous, filterable, liquid concentrate solution of sertraline hydrochloride for oral administration containing sertraline hydrochloride, ethanol, and glycerin and one or more pharmaceutically acceptable excipients. The proposed value of the concentrate was to prepare a formulation with acceptable taste that could be easily swallowed. The '283 patent further discloses a method of using the concentrate whereby the concentrate is diluted with an aqueous diluent prior to administration to a patient. Formulations prepared according to the '283 patent continue to have an objectionable taste but are less objectionable than formulations prepared according to the '518 patent.

Cyclodextrins are well known for their ability to mask the taste of poor tasting compounds. Parent underivatized cyclodextrins and some of their derivatives have been suggested or demonstrated as being useful. Schmidt et al. (Pharmazie. (1993 November) 48(11), 837-41) disclose the use of HP-β-CD in an aqueous oral formulation comprising water and hexetidine, an antimicrobial agent. The formulation reportedly has improved taste in the presence of HP-β-CD. Miyaji et al. (Acta Pharm. Nord., (1992), 4(1), 17-22) disclose aqueous liquid formulations comprising fenbufen with α-CD, β-CD and γ-CD. The formulations reportedly exhibit enhanced bioavailability and reduced bitterness. Han (Zhongguo Zhong Yao Za Zhi. (1990 December), 15(12), 729-31, 765) discloses the formation of a complex between β-CD and bile acid reportedly reducing the bitter taste of the bile acid.

U.S. Pat. No. 5,024,997 to Motola et al. discloses a palatable aqueous ibuprofen solution suitable for oral administration having a pH of about 3 to 5 comprising about 2% to 5% weight ibuprofen by volume of the total composition, about 20% to about 70% weight by volume of at least one taste masking sweetening ingredient and about 22% to about 75% weight by volume of hydroxypropyl beta cyclodextrin having a degree of hydroxpropyl substitution of about 6 to about 7.5, the weight ratio of ibuprofen to hydroxypropyl beta cyclodextrin being 1:11 to 1:15, and water qs to 100% by volume of the composition.

U.S. Pat. No. 5,019,563 to Hunter et al. discloses complexes of β-CD with various salts of ibuprofen. The molar ratio of ibuprofen to β-CD is within the range of from 1:0.20 to 1:0.75. The preferred salt of ibuprofen is the sodium salt. The complexes reportedly have an enhanced taste profile and bioavailability.

However, the ability of a CD to mask the unpleasant taste of compound is highly unpredictable when going from one class of cyclodextrins to another or when going from one drug to another within the same class of cyclodextrins. Therefore, specific combinations of compounds and classes of cyclodextrins are required.

Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds, which can fit all, or part of their structure into these cavities. This process, known as inclusion complexation, may result in increased apparent aqueous solubility and stability for the complexed drug. The complex is stabilized by hydrophobic interactions and does not involve the formation of any covalent bonds.

Chemical modification of the parent cyclodextrins (usually at the hydroxyl moieties) has resulted in derivatives with sometimes improved safety while retaining or improving the complexation ability of the cyclodextrin. Of the numerous derivatized cyclodextrins prepared to date, only two appear to be commercially viable; the 2-hydroxypropyl derivatives (HP-β-CD or HPCD), neutral molecules being commercially developed by Jannsen and others, and the sulfoalkyl ether derivatives (SAE-β-CD or SAE-CD), being developed by CyDex, Inc.

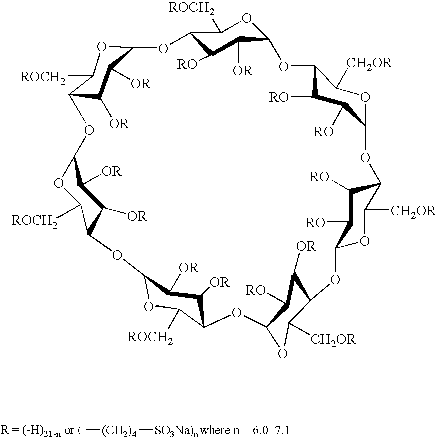

### Sulfobutyl Ether-β-Cyclodextrin (Captisol®)

The SAE-CDs are a class of negatively charged cyclodextrins , which vary in the nature of the alkyl spacer, the salt form, the degree of substitution and the starting parent cyclodextrin. The sodium salt of the sulfobutyl ether derivative of beta-cyclodextrin, with an average of about 7 substituents per cyclodextrin molecule (SBE7-β-CD), is being commercialized by CyDex, Inc. (Kansas) as CAPTISOL® cyclodextrin.

The anionic sulfobutyl ether substituent dramatically improves the aqueous solubility of the parent cyclodextrin. Reversible, non-covalent, complexation of drugs with the CAPTISOL® cyclodextrin generally allows for increased solubility and stability of some drugs in aqueous solutions. However, the improved properties of SAE-CD over HP-β-CD in terms of binding to specific drugs are somewhat unpredictable. Many drugs are known to bind better with SAE-CD, while others are known to bind better with HP-β-CD. Moreover, CAPTISOL® cyclodextrin is relatively new, and its combined use with sertraline HCl for oral administration has not been evaluated or suggested in the prior art.

U.S. Pat. No. 6,267,985 to Chen et al. discloses a method for improving the solubilization of triglycerides and improved delivery of therapeutic agents. The disclosed formulations comprise a combination of two surfactants, a triglyceride and a therapeutic agent that is capable of being solubilized in the triglyceride, the carrier, or both the triglyceride and the carrier. The '985 patent suggests the use of sertraline and of an optional solubilizing agent, such as a cyclodextrin, which can include cyclodextrin derivatives such as hydroxypropyl cyclodextrin (HPCD), sulfobutyl ether cyclodextrin and sulfobutyl ether conjugates of cyclodextrins. HPCD is the preferred cyclodextrin. A taste-masked aqueous liquid oral dosage form comprising SAE-CD, sertraline, and water is not suggested.

U.S. Pat. No. 6,294,192 to Patel et al. discloses triglyceride-free oral pharmaceutical compositions capable of solubilizing therapeutically effective amounts of hydrophobic therapeutic agents. The disclosed formulations include a carrier comprising a combination of a hydrophilic surfactant and a hydrophobic surfactant wherein the composition is substantially free of water and glycerol triesters of selected fatty acids. The '192 patent suggests the use of sertraline and of an optional solubilizing agent, such as a cyclodextrin, which can include cyclodextrin derivatives such as HPCD and sulfobutyl ether cyclodextrin. HPCD is the preferred cyclodextrin. A taste-masked aqueous liquid oral dosage form comprising SAE-CD, sertraline, and water is not suggested.

U.S. Pat. No. 6,383,471 to Chen et al. discloses pharmaceutical compositions, which can be solutions, comprising a hydrophobic therapeutic agent having at least one ionizable functional group, and a carrier. The carrier comprises an ionizing agent capable of ionizing the functional group, a surfactant, and optionally solubilizers, triglycerides, and neutralizing agents. The '471 patent discloses that a cyclodextrin, which can include cyclodextrin derivatives such as hydroxypropyl cyclodextrin (HPCD), sulfobutyl ether cyclodextrin and sulfobutyl ether conjugates of cyclodextrins, can be a suitable solubilizing agent. Sertraline is listed as a drug that can be included in the pharmaceutical composition. A taste-masked aqueous liquid oral dosage form comprising SAE-CD, sertraline, and water is not suggested.

US Patent Application No. 20020192302 to Hsu et al. discloses methods for enhancing the flux of an antidepressant drug through a body surface, by administering an antidepressant drug and a basic permeation enhancer. The pH of the formulations used for the method is claimed to be between 8.0 and 13.0. The '302 application discloses aqueous solutions and discloses sertraline as an example of an antidepressant drug. A second permeation enhancer can be added, including cyclodextrin enhancers. The '302 application does not disclose the use of cyclodextrins or cyclodextrin derivatives for solubilization or taste masking. A taste-masked aqueous liquid oral dosage form comprising SAE-CD, sertraline, and water is not suggested.

US Patent Application No. 20020156066 to Chen et al. disclose a process for preparing a solid dispersion comprising an active ingredient and a water-soluble polymer. The process includes preparing a solution in which an active ingredient and a water-soluble polymer are dissolved in a co-solvent of a volatile organic solvent and water. Sertraline and its acid addition salts are claimed. The '066 application also claims a process wherein the solution is sprayed onto a pharmaceutically acceptable carrier. The claimed carriers include alpha-, beta-, and gamma-cyclodextrins and hydroxypropyl-beta-cyclodextrin. The '066 application discloses but does not claim or teach the use of cyclodextrins as a water soluble polymer in the solution of the process. A taste-masked aqueous liquid oral dosage form comprising SAE-CD, sertraline, and water is not suggested.

US Patent Application No. 20020150616 to Vandecruys discloses pharmaceutical compositions comprising a sparingly water-soluble drug compound, a cyclodextrin, a physiologically tolerable water-soluble acid, and a physiologically tolerable water-soluble organic polymer. The '616 application discloses, but does not teach, the possible use of aqueous compositions, and discloses a preference for substantially water-free compositions. The '616 application discloses sertraline as a sparingly water-soluble drug and physiologically tolerable water-soluble substituted or unsubstituted cyclodextrins. Sulfobutyl ether cyclodextrins are disclosed in the application. A taste-masked aqueous liquid oral dosage form comprising SAE-CD, sertraline, and water is not suggested.

U.S. Pat. No. 6,720,001 to Chen et al., discloses pharmaceutical oil-in-water emulsions for delivery of polyfunctional active ingredients, wherein the emulsions include an aqueous phase, an emulsifier, and an oil phase. Sertraline is claimed as a polyfunctional active ingredient. The '001 patent also claims as an emulsifier, a reaction mixture of a polyol and a fatty acid, glyceride, vegetable oil, hydrogenated vegetable oil, or sterol. Cyclodextrins are disclosed as examples of polyols. A taste-masked aqueous liquid oral dosage form comprising SAE-CD, sertraline, and water is not suggested.

U.S. Patent Application No. 20020012680 to Patel et al. discloses triglyceride-free pharmaceutical compositions comprising a hydrophobic therapeutic agent, and a carrier comprising at least one hydrophilic surfactant and at least one hydrophobic surfactant. The application claims but does not teach the use of sertraline as a suitable hydrophobic therapeutic agent. The claimed formulation can further comprise a solubilizer, which may be a sulfobutyl ether cyclodextrin. A taste-masked aqueous liquid oral dosage form comprising SAE-CD, sertraline, and water is not suggested.

U.S. Pat. No. 6,720,003 to Chen et al. discloses a process for preparing amorphous paroxetine hydrochloride or sertraline hydrochloride. The process comprises the steps of preparing a solution in which paroxetine hydrochloride or sertraline hydrochloride and a water-soluble polymer are dissolved in a co-solvent of a volatile organic solvent and water. The solution is then dried to obtain a composition comprising amorphous paroxetine hydrochloride or sertraline hydrochloride and the water-soluble matrix. Cyclodextrins are suggested for use as the water-soluble polymer or as a carrier onto which the drug-containing solution is sprayed. A taste-masked aqueous liquid oral dosage form comprising SAE-CD, sertraline, and water is not suggested.

U.S. Pat. No. 5,134,127 and No. 5,376,645 to Stella et al. disclose compositions containing an SAE-CD and a drug. Sertraline is not included in the list of drugs that can be used. Moreover, Stella et al. do not suggest a taste-masked aqueous liquid oral dosage form comprising SAE-CD, sertraline, and water.

An oral liquid dosage form of sertraline with an improved taste would be valuable with regard to the issue of non-compliance with treatment, which is believed to affect up to 50% of outpatients and appears to be a particular problem with elderly, pediatric and psychiatric patients (B. Blackwell, Drug Therapy: Patient Compliance, N. Engl. J. Med. 1973, 289(5):249-52). An alcohol free formulation of sertraline would also be valuable as it would eliminate interactions in subjects concurrently taking ANTABUSE® (disulfiram) or other therapeutics having the potential for dangerous or otherwise unacceptable drug interactions with alcohol. A ready-to-use liquid formulation (a formulation not requiring dilution prior to administration) would also be valuable because less manipulation of the dose would be required prior to administration, the purchase and/or availability of a diluting solvent would not be required, and potential chemical and physical interactions of the formulation with the diluent would be eliminated. Therefore, there is a need in the art for an alternative ready-to-use liquid dosage form of sertraline that has acceptable taste and properties.

None of the known art has been able to overcome the disadvantages inherent in the present ZOLOFT® oral concentrate formulation and a need remains for an improved formulation. A need remains for an improved formulation with more acceptable taste, no requirement for dilution prior to use, a reduced potential for interaction with other drugs and formulations known to interact with alcohol, that remains chemically and physically stable under a variety of storage and use conditions, and that is resistant to microbial growth. Additionally, none of the art discloses or suggests the invention as claimed herein.

### SUMMARY OF THE INVENTION

The present invention seeks to overcome some or all of the disadvantages inherent in other known formulations. The invention provides a pharmaceutical composition comprising a taste-masked aqueous oral liquid formulation comprising water, sulfoalkyl ether cyclodextrin (SAE-CD), sertraline (or any pharmaceutically acceptable salt thereof), and optionally one or more pharmaceutically acceptable excipients. The SAE-CD is primarily responsible for masking the taste of the sertraline. Specific pharmaceutically acceptable salts of sertraline include the hydrochloride salt and the mesylate salt. The taste-masked formulation of the invention can be a single-dose or multi-dose formulation. The inventors have also determined that the claimed formulation is also self-preserved against microbial proliferation when the SAE-CD is present in amounts sufficient to stop or reduce the rate of microbial growth once the formulation has become contaminated with a microbe. The present formulation also possesses improved photochemical stability over the ZOLOFT® oral formulation and over other cyclodextrin-based formulations.

In order for the liquid formulation of the invention to be clear, the molar ratio of SAE-CD to sertraline should be at least about 0.98. This molar ratio is sufficient to provide acceptable taste-masking; however, higher molar ratios will result in even further improved taste-masking, since it has been found that taste is improved by increasing the percentage of sertraline bound by SAE-CD. According to specific embodiments, the molar ratio of SAE-CD to sertraline is at least about 1.1:1, 1.5:1, 2.0:1, 5.0: 1,0:1, or 20:1.

The present invention also provides an SAE-CD-based oral solution of sertraline that is pleasant tasting and pharmaceutically stable, and that does not require dilution prior to administration.

Specific embodiments include those wherein: 1) the sulfoalkyl ether cyclodextrin is present in an amount sufficient to provide a clear solution; 2) the sertraline is present in therapeutically effective amounts; 3) the molar ratio of SAE-CD to sertraline is in the range of about 0.95 to 10; 4) sertraline is present at a concentration of about 2-40 mg/mL; 5) SAE-CD is present at a concentration of about 20-700 mg/mL (or 2-70% wt./vol.); 6) the liquid formulation has been prepared by reconstitution of a reconstitutable solid comprising at least SAE-CD and sertraline with an aqueous solution, wherein the reconstitutable solid is as defined herein; 7) the formulation does not require dilution prior to oral administration to a subject; 8) the SAE-CD is sulfobutyl ether 4-β-CD or sulfobutyl ether 7-β-CD; 9) the SAE-CD is a compound of the formula 1 (infra.) or a mixture of compounds thereof; 10) the formulation further comprises a solubilizing agent, a flavoring agent, a sweetening agent, a viscosity inducing agent, an antioxidant, a buffering agent, an acidifying agent, a complexation enhancing agent, a lyophilizing aid (for example, bulking agents or stabilizing agents), an electrolyte, another therapeutic agent, an alkalizing agent, an antimicrobial agent, an antifungal agent or a combination thereof; 11) the liquid formulation is lyophilized or otherwise dehydrated to form a reconstitutable solid; 12) the formulation has a more acceptable taste than the ZOLOFT® oral concentrate formulation, which is nonaqueous and comprises glycerin, alcohol (12%), menthol (flavor) and butylated hydroxytoluene; 13) the formulation has a more acceptable taste than an aqueous formulation not containing a cyclodextrin; 14) the formulation has a more acceptable taste than an aqueous formulation comprising an equivalent molar concentration of another derivatized or underivatized cyclodextrin; 15) the liquid formulation is dilutable with an aqueous based diluent without precipitation of the sertraline; 14) the liquid formulation has improved photochemical stability and undergoes less degradation when exposed to ultraviolet light or fluorescent light as compared to the ZOLOFT® oral concentrate formulation; 15) the liquid formulation is dilutable with commercially available lemon/lime soda, ginger ale, cola, orange juice, or apple juice without significant precipitation; 16) the formulation demonstrates equivalent pharmacokinetics to the ZOLOFT® oral concentrate formulation when administered to a patient; and/or 17) the liquid formulation undergoes less chemical degradation when exposed to ultraviolet light or the light from fluorescent light sources than formulations wherein the SAE-CD has been replaced by equimolar amounts of another cyclodextrin, such as HP-β-CD.

The formulation also has a more acceptable (palatable) taste than the ZOLOFT® oral concentrate formulation when diluted with water, gingerale, lemon/lime soda, lemonade or orange juice.

Another aspect of the invention provides a method for preparing a taste-masked aqueous liquid oral formulation from a reconstitutable solid, the method comprising the steps of:

providing a reconstitutable solid comprising sertraline, SAE-CD and optionally at least one other pharmaceutical excipient, wherein the solid is reconstitutable with an aqueous liquid, and the molar ratio of SAE-CD to sertraline is at least about 0.95 or at least about 0.98; and

reconstituting the solid with a sufficient amount of aqueous liquid carrier sufficient to at least suspend the reconstitutable solid, thereby forming the taste-masked aqueous liquid oral formulation.

Specific embodiments of the invention include those wherein: 1) the liquid formulation is a suspension; 2) the amount of liquid carrier added is sufficient to render the liquid formulation clear; 3) the method further comprises the step of mixing the reconstitutable solid and aqueous liquid carrier; 4) after reconstitution, the liquid formulation is ready for administration to a subject without requiring further dilution; 5) the formulation is a concentrate having a concentration of sertraline in the range from 1 to 110 mg/mL, 2-50 mg/mL or 2-20 mg/mL; 6) the pH of the formulation approximates or is less than the pKa of sertraline; 7) the pH of the formulation is in the range of about 2 to 7.

The invention also provides a method of administering sertraline comprising the step of orally administering a ready-to-use liquid formulation comprising a sulfoalkyl ether cyclodextrin and sertraline or a pharmaceutically acceptable salt thereof.

Specific embodiments of the methods of the invention include those wherein: 1) the liquid formulation is administered orally; 2) the method further comprises the step of diluting a concentrate, according to the invention, with an aqueous liquid carrier prior to administration, thereby providing the ready-to-use liquid formulation; 3) the method further comprises the step of forming the liquid formulation by mixing an aqueous liquid carrier with a reconstitutable solid according to the invention; 4) the liquid formulation is formulated as described herein; 5) the liquid formulation causes less or no undesirable pharmacological interaction with disulfiram or other pharmacologically active agents known to have undesirable interactions with alcohol as compared to the ZOLOFT® oral concentrate formulation; 6) the liquid formulation provides equivalent or improved chemical stability characteristics as compared to the ZOLOFT® oral concentrate formulation; and/or 7) the liquid formulation provides a pharmacokinetic and/or pharmacodynamic profile similar to that of the ZOLOFT® oral concentrate formulation.

The present invention also provides a method of treating or preventing diseases or conditions that are caused by disorders of the serotonergic system, the method comprising the step of orally administering the aqueous solution of the invention to a patient in need thereof. Specific embodiments of the invention include those wherein: 1) the disease or conditions is selected from the group consisting of depression, anorexia, chemical dependencies, anxiety-related disorders (such as panic disorder, obsessive-compulsive disorder, generalized anxiety disorder, phobias, post traumatic stress disorder and avoidant personality disorder), premature ejaculation, cancer and post myocardial infarction; 2) the formulation is administered according to the dosage and administration practices for ZOLOFT® oral concentrate.

The present invention also provides methods of preparing an SAE-CD-based aqueous solution of sertraline or a pharmaceutically acceptable salt thereof.

Another aspect of the invention provides a kit comprising a first pharmaceutical composition comprising SAE-CD and a second pharmaceutical composition comprising sertraline or a pharmaceutically acceptable salt thereof.

Other features, advantages and embodiments of the invention will become apparent to those skilled in the art by the following description, accompanying examples.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts data obtained from a room temperature phase solubility study conducted with sertraline hydrochloride and SBE7-β-CD, gamma-CD, or 2-hydroxypropyl-β-CD in water

FIG. 2 depicts the concentration of sertraline in the plasma of human subjects after dosing with sertraline-containing formulations.

FIG. 3 depicts the solubility of sertraline HCl in solutions containing 0, 10 or 20% w/v sulfobutylether-7-β-cyclodextrin (Captisol®) at various pH values.

### DETAILED DESCRIPTION OF THE INVENTION

A formulation according to the invention comprising sertraline or a pharmaceutically acceptable salt thereof and a sulfoalkyl ether cyclodextrin overcomes some or all known disadvantages present in prior art formulations of sertraline. The present formulation is substantially free of any purposefully-added ethyl alcohol, is physically and chemically stable, and has an improved taste as compared to commercially available non-cyclodextrin-based aqueous liquid oral dosage forms and other cyclodextrin-based aqueous liquid oral dosage forms. When prepared in ready-to-use (i.e., ready-to-administer) form, the liquid formulation of the invention does not require dilution prior to administration. Moreover, the present formulation exhibits substantially equivalent pharmacokinetics to the ZOLOFT® oral concentrate formulation when administered orally to patients. When present as a concentrate, the present formulation is also dilutable in a broad range of aqueous based diluents without formation of precipitate.

As used herein and unless otherwise specified, the term “sertraline” includes all neutral, free base, salt, crystalline, non-crystalline, amorphous and/or polymorphic forms of the same. The sertraline can be present in anhydrous or hydrated form prior to use in present formulation. The preferred salt of sertraline is a pharmaceutically acceptable salt. As used herein, “pharmaceutically acceptable salt” refers to derivatives of sertraline wherein the active agent is modified by reacting it with an acid as needed to form an ionically bound pair. Examples of pharmaceutically acceptable salts include conventional non-toxic salts or the quaternary ammonium salts of the parent compound formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. Other salts are prepared from organic acids such as amino acids, acetic, propionic, butyric, succinic, glycolic, gluconic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethanesulfonic, benzenesulfonic, oxalic, isethionic, and other acids known to those of ordinary skill in the art. Lists of other suitable salts are found in Remington's Pharmaceutical Sciences, 17^{th}. ed., Mack Publishing Company, Easton, Pa., 1985, the relevant disclosure of which is hereby incorporated by reference.

As used herein, the term reconstitutable solid (reconstitutable composition) is taken to mean a solid capable of dissolution in an aqueous liquid medium to form a reconstituted liquid, wherein after dissolution the liquid medium is suitable for administration. In one embodiment, the reconstitutable solid forms a taste-masked liquid formulation that is visibly clear. In another embodiment, the liquid formulation is a taste-masked suspension. A reconstitutable pharmaceutical formulation according to the present invention comprises sertraline, SAE-CD and optionally, at least one other pharmaceutical excipient, wherein the molar ratio of SAE-CD to sertraline is as defined herein. A reconstitutable solid can be prepared by removal of the liquid medium from an aqueous liquid solution comprising SAE-CD and sertraline, and optionally other components to form the solid. The reconstitutable solid composition can comprise an admixture of a solid SAE-CD and a sertraline-containing solid and optionally at least one other pharmaceutical excipient, such that a major portion of the sertraline is not complexed with the SAE-CD prior to reconstitution. Alternatively, the composition can comprise a solid mixture of an SAE-CD, sertraline and optionally at least one other pharmaceutical excipient, wherein a major portion of the sertraline is complexed with the SAE-CD prior to reconstitution. A reconstitutable solid will generally comprise less than 8% wt. water. The reconstitutable solid formulation provides equivalent or improved chemical stability of sertraline as compared to the marketed ZOLOFT® oral concentrate formulation. This composition is reconstituted with an aqueous solution to form a liquid formulation containing sertraline and other agents that can be administered orally to a subject. The liquid formulation used in the preparation of a reconstitutable formulation may be prepared as described herein for the diluted or concentrated liquid formulations. It may also be prepared to contain an SAE-CD and the sertraline at concentrations greater than typically used in the liquid formulation of the invention, while maintaining the same SAE-CD to sertraline molar ratio. Applicants note that any composition according to the invention can be dissolved or diluted with another liquid containing SAE-CD.

The reconstitutable composition can be prepared according to any of the following processes. A liquid formulation of the invention is first prepared, then a solid is formed by lyophilization (freeze-drying), spray drying, spray freeze-drying, vacuum-drying, antisolvent precipitation, various processes utilizing supercritical or near supercritical fluids, or other methods known to those of ordinary skill in the art of the liquid formulation to make a powder or a solid suitable for reconstitution. As noted above, the reconstitutable solid can be an admixture of the dry components, which is prepared by physically blending the components in the absence of excess moisture, i.e. the moisture should be less than about 60% RH.

A reconstitutable solid can be a powder, glassy solid, porous solid, granulate, pellet, bead, compressed solid or particulate.

As used in regards to an SAE-CD-containing composition or formulation according to the invention, the term dilutable refers to a liquid formulation containing SAE-CD and sertraline, wherein the formulation can be further diluted with a clear aqueous liquid carrier at room temperature, e.g., ambient temperature such as a temperature of about 20°-28° C., preferably without significant precipitation of sertraline, i.e. if precipitation occurs it is less than or equal to about 3% wt. of sertraline, while providing a final clear solution when diluted to a sertraline concentration of about 0.15 to 5 mg/mL. When a dilutable SAE-CD and sertraline-containing formulation is diluted with a non-clear solution, the resulting mixture may or may not be clear. A dilutable SAE-CD and sertraline-containing liquid can be diluted with another solution that does not contain SAE-CD, and the resulting diluted solution will have a lower concentration of solubilized sertraline preferably without causing significant precipitation of sertraline.

Exemplary liquids for diluting a liquid formulation of the invention include commercially available beverages such as carbonated beverages, non-carbonated beverages, and juices. Exemplary carbonated beverages include flavored and non-flavored sodas, wherein the flavor is a cola, lemon, lime, root beer, bubble gum, cherry, orange and other flavors or mixtures thereof. Exemplary juices include apple, lemon, lime, orange, grape, cherry, cranberry, grapefruit, strawberry, kiwi, raspberry, blueberry, blackberry, dewberry, tangerine, pineapple, watermelon, cantaloupe, ginger, guava, mango, papaya, plum, apricot, pear, peach, nectarine, pomegranate, and other juices or mixtures thereof. Accordingly, an SAE-CD and sertraline-containing solution that is not dilutable according to the invention will form a significant amount (>3% wt. of active agent) of precipitate when diluted with another solution.

It should be noted that a solution that is not dilutable with water at room temperature may be rendered dilutable with an aqueous solution that contains SAE-CD as long as the final molar ratio of sertraline to SAE-CD in the diluted solution is within the required range as described herein. The invention therefore provides a method of rendering dilutable a previously non-dilutable (as defined herein) sertraline-containing solution comprising the step of diluting the previously non-dilutable solution with a second solution containing SAE-CD such that the molar ratio of SAE-CD to sertraline in the diluted solution is as defined herein.

Temperature may have an effect upon the dilutability of a solution. In general, the determination of whether or not a solution is dilutable is made at approximately 25° C. or ambient temperature, e.g., 20°-28° C. A solution that is not dilutable at about 25° C. can be made dilutable with water at room temperature by dilution at an elevated temperature, such as >30° C., >40° C., >50° C. or higher. This heated dilution can be performed by diluting the first 25° C. solution with a heated solution or by mixing and heating two solutions which are initially at ambient temperature. Alternatively, the two solutions can be heated separately and then mixed.

Dilutability of an SAE-CD and sertraline-containing solution at ambient temperature is particularly important in the clinical setting wherein solutions are not typically heated prior to mixing. Accordingly, the present invention provides solutions of sertraline that can be diluted at ambient temperature without the need of a surfactant, organic solvent, soap, detergent or other such compound.

As used herein, a pharmaceutically acceptable liquid carrier is any aqueous medium used in the pharmaceutical sciences for dilution or dissolution of oral or peroral formulations.

The formulation of the invention comprises sertraline and a sulfoalkyl ether cyclodextrin of the formula 1: 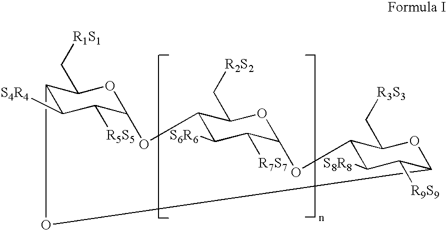
wherein:

n is 4, 5 or 6;

R₁, R₂, R₃, R₄, R₅, R₆, R₇, R₈ and R₉ are each, independently, —O— or a —O-(C₂-C₆ alkylene)-SO₃⁻ group, wherein at least one of R₁ and R₂ is independently a —O-(C₂-C₆ alkylene)-SO₃⁻ group, preferably a —O—(CH₂)ₘSO₃— group, wherein m is 2 to 6, preferably 2 to 4, (e.g. —OCH₂CH₂CH₂SO₃— or —OCH₂CH₂CH₂CH₂SO₃⁻); and

S₁, S₂, S₃, S₄, S₅, S₆, S₇, S₈ and S₉ are each, independently, a pharmaceutically acceptable cation which includes, for example, H⁺, alkali metals (e.g. Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine and (C₄-C₈)-cycloalkanolamine.

The SAE-CD used in the liquid or solid formulation is described in U.S. Pat. No. 5,376,645 and No. 5,134,127 to Stella et al, the entire disclosures of which are hereby incorporated by reference. The preparation process may comprise dissolving the cyclodextrin in aqueous base at an appropriate temperature, e.g., 70° to 80° C., at the highest concentration possible. For example, to prepare the cyclodextrin derivatives herein, an amount of an appropriate alkyl sultone, corresponding to the number of moles of primary CD hydroxyl group present, is added with vigorous stirring to ensure maximal contact of the heterogeneous phase.

The terms “alkylene” and “alkyl,” as used herein (e.g., in the —O—(C₂-C₆-alkylene)SO₃⁻ group or in the alkylamines), include linear, cyclic, and branched, saturated and unsaturated (i.e., containing one double bond) divalent alkylene groups and monovalent alkyl groups, respectively. The term “alkanol” in this text likewise includes both linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

Exemplary SAE-CD derivatives include SBE4-β-CD, SBE7-β-CD (CAPTISOL® cyclodextrin), SBE11-β-CD, SBE5-γ-CD, SBE9-γ-CD which correspond to SAE-CD derivatives of the Formula 1 wherein n=5, 5, 5, 6 and 6, resepectively; m is 4; and there are on average 4, 7, 11, 5 and 9 sulfoalkyl ether substituents present, respectively. It has been found that these SAE-CD derivatives increase the solubility of poorly water soluble drugs, such as sertraline, to varying degrees in ways that have not been suggested or disclosed by the prior art.

Other exemplary SAE-CD derivatives include those of the formula SAEx-R-CD (Formula 2), wherein SAE is sulfomethyl ether (SME), sulfoethyl ether (SEE), sulfopropyl ether (SPE), sulfobutyl ether (SBE), sulfopentyl ether (SPtE), or sulfohexyl ether (SHE); x (average or specific degree of substitution) is 1- 18, 1-21, 1-24, when R (ring structure of parent cyclodextrin) is α, β or γ, respectively, and CD is cyclodextrin.

The present invention provides compositions containing a mixture of cyclodextrin derivatives, having the structure set out in formula (1), where the composition overall contains on the average at least 1 and up to 3n +6 alkylsulfonic acid moieties per cyclodextrin molecule. The present invention also provides compositions containing a single type of cyclodextrin derivative, or at least 50% of a single type of cyclodextrin derivative.

It should be understood that other SAE-CD compounds of the formula 1 may be used in the liquid formulation of the invention. These other SAE-CD formulations differ from SBE7-β-CD in their degree of substitution by sulfoalkyl groups, the number of carbons in the sulfoalkyl chain, their molecular weight, the number of glucopyranose units contained in the base cyclodextrin used to form the SAE-CD and or their substitution patterns. In addition, the derivatization of β-cyclodextrin with sulfoalkyl groups occurs in a controlled, although not exact manner. For this reason, the degree of substitution is actually a number representing the average number of sulfoalkyl groups per cyclodextrin (for example, SBE7-β-CD, has an average of 7 substitutions per cyclodextrin). In addition, the regiochemistry of substitution of the hydroxyl groups of the cyclodextrin is variable with regard to the substitution of specific hydroxyl groups of the hexose ring. For this reason, sulfoalkyl substitution of the different hydroxyl groups is likely to occur during manufacture of the SAE-CD, and a particular SAE-CD will possess a preferential, although not exclusive or specific, substitution pattern. Given the above, the molecular weight of a particular SAE-CD may vary from batch to batch and will vary from SAE-CD to SAE-CD. All of these variations can lead to changes in the complexation equilibrium constant which in turn will affect the required molar ratios of the SAE-CD to sertraline. The equilibrium constant is also somewhat variable with temperature and allowances in the ratio are required such that the agent remains solubilized during the temperature fluctuations that can occur during manufacture, storage, transport, and use. The equilibrium constant is also variable with pH and allowances in the ratio are required such that the agent remains solubilized during pH fluctuations that can occur during manufacture, storage, transport, and use. The equilibrium constant is also variable by the presence of other excipients (e.g., buffers, preservatives, antioxidants). Accordingly, the ratio of SAE-CD/sertraline may need to be varied (±) from the ratios set forth herein in order to compensate for the above-mentioned variables.

The cyclodextrin derivatives of the present invention are obtained as purified compositions, i.e., compositions containing at least 90 wt. % or 95 wt. % of cyclodextrin derivative(s) in terms of the total amount of cyclodextrin present, the balance of cyclodextrin comprising unreacted parent cyclodextrin. In a preferred embodiment, purified compositions containing at least 98 wt. % cyclodextrin derivative(s) are obtained. In some of the compositions of the invention unreacted cyclodextrin has been substantially removed, with the remaining impurities (i.e., <5 wt. % of composition) being inconsequential to the performance of the cyclodextrin derivative-containing composition.

According to other embodiments, the amount of unreacted parent cyclodextrin present in the SAE-CD is up to about 50% wt. of the SAE-CD, less than about 40% wt., less than 30% wt., or less than 20% wt. based upon the total dry weight of cyclodextrin.

By “sertraline/SAE-CD complex” is generally meant a clathrate or inclusion complex of a sulfoalkyl ether cyclodextrin derivative of the formula (1) and sertraline. The complex can be a binary or ternary complex (the salt form of sertraline is complexed). The ratio of SAE-CD:sertraline present in the molecular complex can vary and can be in the range of about 0.95 to 750, on a molar basis. In another embodiment of the dosage forms described herein, the ratio of SAE-CD:sertraline is in the range of about 0.95 to about 20 on a molar basis. Thus, the SAE-CD will generally be, but need not be, present in excess of the sertraline. The amount of excess will be determined by the intrinsic solubility of the agent, the expected dose of the agent, and the binding constant for inclusion complexation between the specific drug (agent) and the specific SAE-CD.

By “major portion” is meant at least about 50% by weight of the therapeutic compound. In various specific embodiments, greater than 50%, 60%, 75%, 90% or 95% by weight of the sertraline can be complexed with an SAE-CD while in the pharmaceutical formulation. The actual percent of drug that is complexed will vary according to the complexation equilibrium constant characterizing the complexation of a specific SAE-CD to sertraline and to the concentrations of SAE-CD and sertraline available for complexation. At a constant molar ratio of SAE-CD:sertraline, the free fraction of sertraline increases as the concentration of SAE-CD and sertraline decreases. Free fraction refers to the amount of uncomplexed sertraline in a solution containing SAE-CD. The free fraction of sertraline should be minimized in order to enhance taste-masking. At lower concentrations, such as at 5 mg sertraline/mL, and at an SAE-CD:sertraline molar ratio of 0.95, the free fraction of sertraline is about 25% (about 1.25 mg/mL). At high concentrations, such as at 64 mg sertraline/mL and at an SAE-CD:sertraline molar ratio of 0.98, the free fraction of sertraline is about 8% (about 5.5 mg/mL). For example, the formulation of Example 7 contained sertraline (20 mg/mL), SBE7-β-CD (17% wt./vol), water and an SAE-CD:sertraline molar ratio of about 1.3. That formulation, which had acceptable taste-masking, has a free fraction of sertraline of about 5% (1.0 mg/mL).

Accordingly, the SAE-CD should be present in the formulation in an amount sufficient to minimize the free fraction (concentration) of sertraline to the extent that the taste of the formulation is acceptable. In general, the concentration of free sertraline should be less than about 2.0 mg/mL, less than about 1.5 mg/mL, less than about 1.0 mg/mL, less than about 0.5 mg/mL, less than 0.1 mg/mL, less than 0.05 mg/mL, less than 0.005 mg/mL.

FIG. 1 depicts a phase solubility curve for the binding of sertraline to SBE7-β-CD, γ-CD or HP-β-CD (without adjusting the pH) at about 25° C. At lower molar concentrations of cyclodextrin (less than about 0.08 M), the phase solubility curve for each cyclodextrin is very similar. As the concentration of cyclodextrin and sertraline increases, the SBE7-β-CD and HP-β-CD outperform the γ-CD.

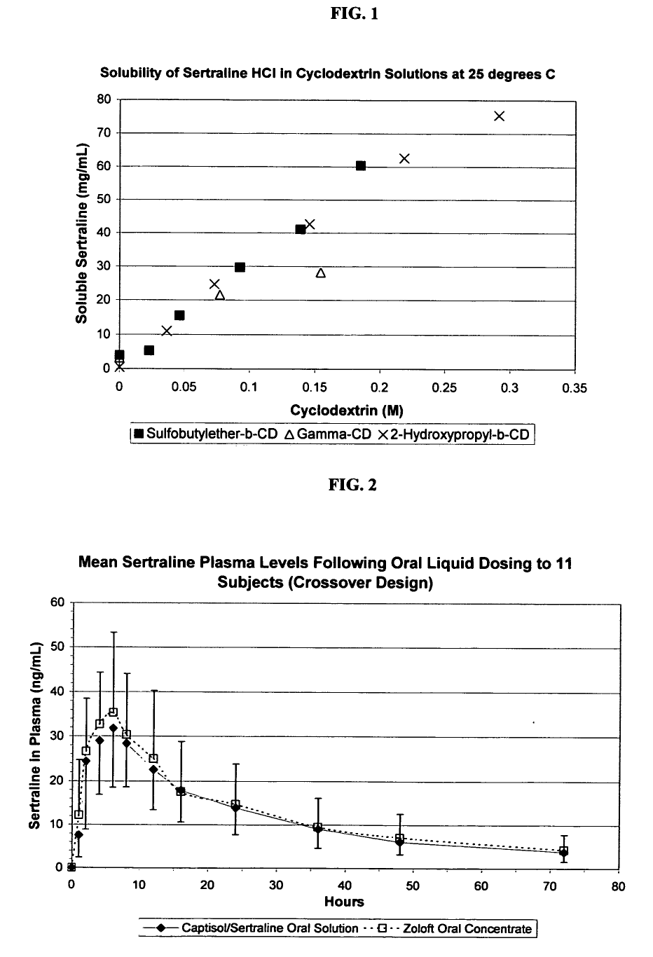

A ready-to-use formulation was prepared according to Example 7 and administered orally to patients without dilution prior to administration. For comparison, the ZOLOFT® oral concentrate formulation was also administered to the patients. A fourteen day washout period was used between dosings. The plasma concentration of sertraline in the patients was monitored for a period of about 72 hours after dosing. FIG. 2 depicts the plasma concentration profile for sertraline after administration of the formulations to the patients. The data demonstrate that, in terms of pharmacokinetics, the SAE-CD based formulation is substantially equivalent to the ZOLOFT® oral concentrate formulation. The pharmacokinetic data is summarized in greater detail in Example 9.

Subjects evaluated the taste of the SAE-CD based formulation and the ZOLOFT® formulation according to the method of Example 10. The formulation of the invention significantly out performed the ZOLOFT® formulation. Another taste test was performed to compare the taste-masking of SAE-CD to that of HP-β-CD. Since, as noted above, the inventors have found that HP-β-CD and CAPTISOL® possess about the same binding constant for sertraline under the conditions tested, it was initially assumed that both would provide substantially the same level of taste-masking. Surprisingly, the SAE-CD provided improved taste-masking over HP-β-CD.

Additional studies were conducted to evaluate Na-SAE-CD (sodium salt of SAE-CD), HP-β-CD and γ-CD. Na-SAE-CD significantly out performed HP-β-CD (Example 10) and γ-CD even at low concentrations of CD, where the binding constants for sertraline were similar.

As noted above, performance of SAE-CD, in terms of taste-masking, may vary according to the particular counterion for the sulfonate group. The sodium, calcium and ammonium salt forms of SAE-CD were evaluated and determined to provide taste-masking. The sodium salt provided the greatest level of taste-masking under the test conditions employed.

The photochemical stability of two SAE-CD based formulations (Formulations B and C), an HP-β-CD based formulation (Formulation A), a γ-CD based formulation (Formulation D), and the ZOLOFT® oral concentrate formulation (Formulation E) were evaluated as detailed in Example 8. A portion of each formulation was exposed to ultraviolet light or fluorescent light over a period of fifteen days. At time points “0-days” and “15-days”, aliquots of solution were withdrawn and analyzed by HPLC to determine their impurity profile. Any new peaks that appeared in the chromatograms were designated as corresponding to degradants formed during storage. SAE-CD out performed both of the other cyclodextrins as well as the ZOLOFT® oral concentrate formulation. A lower number of degradants were formed in and a lower amount of the degradants was obtained in the SAE-CD containing formulation. It is surprising that SAE-CD would out perform the other two cyclodextrins given the similarity in binding constants of those other cyclodextrins for sertraline. Both SBE4-β-CD and SBE7-β-CD exhibited improved photochemical stability over the other formulations.

The present formulations comprising SAE-CD were evaluated according to Example 14 to determine whether or not they could be preserved even though a conventional preservative added to the formulation might be bound by the SAE-CD. The results indicate that formulations of sertraline prepared according to the invention possess microbial growth retarding or preservative properties and pass the criteria set forth in the USP and the EP for preserved oral solutions. In other words, an aqueous liquid formulation as prepared herein can be preserved, at least with regard to the microbes tested and under the test conditions employed. The data are summarized in Example 14.

The package insert for the ZOLOFT® oral concentrate indicates that the formulation must be diluted with a beverage prior to administration. The dilution, however, is problematic as preciptation of ZOLOFT® often follows. The dilutability of the present formulations with a number of different beverages was evaluated and compared to the dilutability of the ZOLOFT® formulation under the same conditions. The evaluation was conducted as detailed in Example 15. The results are shown in the following table.

| | Time | Cyclodextrin | |
|---|---|---|---|
| Diluent | (min) | Formulation | ZOLOFT ® Oral Concentrate |
| Water | 5 | Clear and colorless | Very cloudy, white fine |
| | | | suspension |
| | 30 | Slight white haze | Very cloudy, white fine |
| | | | suspension |
| Lemon/ | 5 | Clear and colorless | Clear and colorless |
| lime | 30 | Clear and colorless | Clear and colorless |
| soda | | | |
| Ginger | 5 | Clear and original color | Cloudy, original color |
| ale | 30 | Clear and original color | Cloudy, original color |
| Apple | 5 | Clear and original color | Cloudy, original color |
| juice | 30 | Clear and original color | Cloudy, original color |
| Orange | 5 | No visible precipitate | No visible precipitate |
| juice | 30 | No visible precipitate | No visible precipitate |
| Cola | 5 | Clear and original color | Brown cloudy precipitate at |
| | | | bottom of clear colorless |
| | | | solution |
| | 30 | Clear and original color | Brown cloudy precipitate at |
| | | | bottom of clear colorless |
| | | | solution |
| Lemon- | 5 | Clear and original color | Cloudy with white floating |
| ade | | | precipitate |
| | 30 | Clear and original color | Cloudy with white floating |
| | | | precipitate |

In almost every single case, no significant precipitation was observed after dilution of the cyclodextrin formulation of the invention with the indicated beverages. On the other hand, the ZOLOFT® oral concentrate exhibited significant precipitation in almost every case tested. Accordingly, the invention provides a clear aqueous oral liquid formulation of sertraline that is stable to dilution with common beverages.

The chemical stability of the liquid formulations of the invention, in terms of formation of a precipitate, can be enhanced by adjusting the pH of the liquid carrier. The chemical stability can also be enhanced by converting the liquid formulation to a solid or powder formulation.

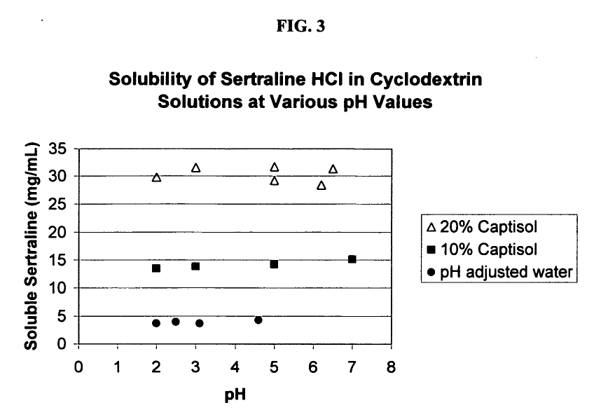

The pH of the liquid formulation will generally range from about pH 3.0 to about pH 7.0; however, liquid formulations having higher or lower pH values can also be prepared. FIG. 3 depicts the results of a study to determine the effect of solution pH upon the solubility of sertraline in solutions containing varying amounts of SAE-CD. The results show that the solubility of the drug is independent of the pH over the range evaluated. The solubilization of sertraline by the cyclodextrin is dependent on the cyclodextrin content but not pH over this same range.

The invention also provides a pharmaceutical kit comprising a first container containing a liquid vehicle and a second container containing a reconstitutable solid pharmaceutical composition as described above. The liquid vehicle comprises an aqueous liquid carrier such as water, dextrose, saline, lactated Ringer's solution, or any other pharmaceutically acceptable aqueous liquid vehicles for the preparation of a liquid pharmaceutical compound.

Although not necessary, the formulation of the present invention may include a antioxidant, acidifying agent, alkalizing agent, buffering agent, bulking agent, cryoprotectant, density modifier, electrolyte, flavors, fragrance, glucose, stabilizer, plasticizer, solubility-enhancing agent, sweeteners, surface tension modifier, volatility modifier, viscosity modifier, other excipients known by those of ordinary skill in the art for use in preserved formulations, or a combination thereof.

A complexation-enhancing agent can be added to the aqueous liquid formulation of the invention. A complexation-enhancing agent is a compound, or compounds, that enhance(s) the complexation of sertraline with the SAE-CD. When the complexation-enhancing agent is present, the required ratio of SAE-CD to sertraline may need to be changed such that less SAE-CD is required. Suitable complexation enhancing agents include one or more pharmacologically inert water soluble polymers, hydroxy acids, and other organic compounds typically used in liquid formulations to enhance the complexation of a particular agent with cyclodextrins. Suitable water soluble polymers include water soluble natural polymers, water soluble semisynthetic polymers (such as the water soluble derivatives of cellulose) and water soluble synthetic polymers. The natural polymers include polysaccharides such as inulin, pectins, algin derivatives and agar, and polypeptides such as casein and gelatin. The semi-synthetic polymers include cellulose derivatives such as methylcellulose, hydroxyethyl cellulose, hydroxypropyl cellulose, their mixed ethers such as hydroxypropyl methylcellulose and other mixed ethers such as hydroxyethyl ethylcellulose, hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Suitable hydroxy acids include by way of example, and without limitation, citric acid, malic acid, lactic acid, and tartaric acid and others known to those of ordinary skill in the art.

Hydrophilic polymers can be used to improve the performance of formulations containing a cyclodextrin. Loftsson has disclosed a number of polymers suitable for combined use with a cyclodextrin (underivatized or derivatized) to enhance the performance and/or properties of the cyclodextrin. Suitable polymers are disclosed in Pharmazie (2001), 56(9), 746-747; International Journal of Pharmaceutics (2001), 212(1), 29-40; Cyclodextrin: From Basic Research to Market, International Cyclodextrin Symposium, 10th, Ann Arbor, Mich., United States, May 21-24, 2000 (2000), 10-15 (Wacker Biochem Corp.: Adrian, Mich.); PCT International Publication No. WO 9942111; Pharmazie, 53(11), 733-740 (1998); Pharm. Technol. Eur., 9(5), 26-34 (1997); J. Pharm. Sci. 85(10), 1017-1025 (1996); European Patent Application EP0579435; Proceedings of the International Symposium on Cyclodextrins, 9th, Santiago de Comostela, Spain, May 31-Jun. 3, 1998 (1999), 261-264 (Editor(s): Labandeira, J. J. Torres; Vila-Jato, J. L. Kluwer Academic Publishers, Dordrecht, Neth); S.T.P. Pharma Sciences (1999), 9(3), 237-242; ACS Symposium Series (1999), 737 (Polysaccharide Applications), 24-45; Pharmaceutical Research (1998), 15(11), 1696-1701; Drug Development and Industrial Pharmacy (1998), 24(4), 365-370; International Journal of Pharmaceutics (1998), 163(1-2), 115-121; Book of Abstracts, 216th ACS National Meeting, Boston, Aug. 23-27 (1998), CELL-016, American Chemical Society; Journal of Controlled Release, (1997), 44/1 (95-99); Pharm. Res. (1997) 14(11), S203; Investigative Ophthalmology & Visual Science, (1996), 37(6), 1199-1203; Proceedings of the International Symposium on Controlled Release of Bioactive Materials (1996), 23rd, 453-454; Drug Development and Industrial Pharmacy (1996), 22(5), 401-405; Proceedings of the International Symposium on Cyclodextrins, 8th, Budapest, Mar. 31-Apr. 2, (1996), 373-376. (Editor(s): Szejtli, J.; Szente, L. Kluwer: Dordrecht, Neth.); Pharmaceutical Sciences (1996), 2(6), 277-279; European Journal of Pharmaceutical Sciences, (1996) 4(SUPPL.), S144; Third European Congress of Pharmaceutical Sciences Edinburgh, Scotland, UK Sep. 15-17, 1996; Pharmazie, (1996), 51(1), 39-42; Eur. J. Pharm. Sci. (1996), 4(Suppl.), S143; U.S. Pat. No. 5,472,954 and No. 5,324,718; International Journal of Pharmaceutics (Netherlands), (Dec. 29, 1995) 126, 73-78; Abstracts of Papers of the American Chemical Society, (02 Apr. 1995) 209(1), 33-CELL; European Journal of Pharmaceutical Sciences, (1994) 2, 297-301; Pharmaceutical Research (New York), (1994) 11(10), S225; International Journal of Pharmaceutics (Netherlands), (Apr. 11, 1994) 104, 181-184; and International Journal of Pharmaceutics (1994), 110(2), 169-77, the entire disclosures of which are hereby incorporated by reference.

Other suitable polymers are well-known excipients commonly used in the field of pharmaceutical formulations and are included in, for example, Remington's Pharmaceutical Sciences, 18th Edition, Alfonso R. Gennaro (editor), Mack Publishing Company, Easton, Pa., 1990, pp. 291-294; Alfred Martin, James Swarbrick and Arthur Commarata, Physical Pharmacy. Physical Chemical Principles in Pharmaceutical Sciences, 3rd edition (Lea & Febinger, Philadelphia, Pa., 1983, pp. 592-638); A. T. Florence and D. Altwood, (Physicochemical Principles of Pharmacy, 2nd Edition, MacMillan Press, London, 1988, pp. 281-334. The entire disclosures of the references cited herein are hereby incorporated by references. Still other suitable polymers include water-soluble natural polymers, water-soluble semi-synthetic polymers (such as the water-soluble derivatives of cellulose) and water-soluble synthetic polymers. The natural polymers include polysaccharides such as inulin, pectin, algin derivatives (e.g. sodium alginate) and agar, and polypeptides such as casein and gelatin. The semi-synthetic polymers include cellulose derivatives such as methylcellulose, hydroxyethylcellulose, hydroxypropyl cellulose, their mixed ethers such as hydroxypropyl methylcellulose and other mixed ethers such as hydroxyethyl ethylcellulose and hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Other natural, semi-synthetic and synthetic polymers not named here which meet the criteria of water solubility, pharmaceutical acceptability and pharmacological inactivity are likewise considered to be within the ambit of the present invention.

A solubility-enhancing agent can be added to the aqueous liquid formulation of the invention. A solubility-enhancing agent is a compound, or compounds, that enhance(s) the solubility of sertraline in the liquid formulation. When a complexation-enhancing agent is present, the ratio of SAE-CD to sertraline may need to be changed such that less SAE-CD is required. Suitable solubility enhancing agents include one or more organic solvents, detergents, soaps, surfactants and other organic compounds typically used in oral solution formulations to enhance the solubility of a particular agent. Suitable organic solvents include, for example, ethanol, glycerin, polyethylene glycols, propylene glycol, poloxomers, and others known to those of ordinary skill in the art.

As used herein, the term “flavor” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available strawberry, orange, grape, cherry, vanilla, mint and citrus flavors and mixtures thereof. The amount of flavoring may depend on a number of factors, including the organoleptic effect desired. Flavors will be present in any amount as desired by those of ordinary skill in the art. Particularly flavors are the strawberry and cherry flavors and citrus flavors such as orange.

As used herein, the term “sweetener” is intended to mean a compound used to impart sweetness to a preparation. Such compounds include, by way of example and without limitation, aspartame, dextrose, glycerin, mannitol, saccharin sodium, sorbitol, xylitol, fructose, high fructose corn syrup, maltodextrin, sucralose, sucrose, other materials known to one of ordinary skill in the art, and combinations thereof.

As used herein, a fragrance is a relatively volatile substance or combination of substances that produces a detectable aroma, odor or scent. Exemplary fragrances include those generally accepted as FD&C.

As used herein, the term “alkalizing agent” is intended to mean a compound used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “preservative” is intended to mean a compound used to prevent the growth of microorganisms. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl, or butyl parabens and others known to those of ordinary skill in the art.

As used herein, the term “antioxidant” is intended to mean an agent which inhibits oxidation and thus is used to prevent the deterioration of preparations by the oxidative process. Such compounds include by way of example and without limitation, acetone, sodium bisulfate, ascorbic acid, ascorbyl palmitate, citric acid, butylated hydroxyanisole, butylated hydroxytoluene, hydrophosphorous acid, monothioglycerol, propyl gallate, sodium ascorbate, sodium citrate, sodium sulfide, sodium sulfite, sodium bisulfite, sodium formaldehyde sulfoxylate, thioglycolic acid, sodium metabisulfite, EDTA (edetate), pentetate and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, monobasic sodium acetate, sodium bicarbonate, sodium tartrate and sodium citrate anhydrous and dihydrate and others known to those of ordinary skill in the art.

As used herein, the term “stabilizer” is intended to mean a compound used to stabilize a therapeutic agent against physical, chemical, or biochemical process that would otherwise reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art.

As used herein, the term “viscosity modifier” is intended to mean a compound or combination of compounds capable of increasing or decreasing the viscosity of the liquid formulation. Some of the polymers disclosed herein can be used as viscosity modifiers.

As used herein, the term “tonicity modifier” is intended to mean a compound or compounds that can be used to adjust the tonicity of the liquid formulation. Suitable tonicity modifiers include glycerin, lactose, mannitol, dextrose, sodium chloride, sodium sulfate, sorbitol, trehalose and others known to those or ordinary skill in the art.

As used herein, the term “antifoaming agent” is intended to mean a compound or compounds that prevents or reduces the amount of foaming that forms on the surface of the liquid formulation. Suitable antifoaming agents include by way of example and without limitation, dimethicone, simethicone, octoxynol and others known to those of ordinary skill in the art.

As used herein, the term “bulking agent” is intended to mean a compound used to add bulk to the reconstitutable solid and/or assist in the control of the properties of the formulation during preparation. Such compounds include, by way of example and without limitation, dextran, trehalose, sucrose, polyvinylpyrrolidone, lactose, inositol, sorbitol, dimethylsulfoxide, glycerol, albumin, calcium lactobionate, and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

It should be understood, that compounds used in the pharmaceutical arts generally serve a variety of functions or purposes. Thus, if a compound named herein is mentioned only once or is used to define more than one term herein, its purpose or function should not be construed as being limited solely to that named purpose(s) or function(s).

The liquid formulation of the invention can be prepared by numerous different methods. According to one method, a first aqueous solution comprising SAE-CD is prepared. Then, a second solution comprising sertraline is prepared. Finally, the first and second solutions are mixed to form the liquid formulation. The first and second solutions can independently comprise other excipients and agents described herein. Additionally, the second solution can be water and/or an organic solvent-base solution. Another method of preparation is similar to the above-described method except that the sertraline is added directly to the first solution without the formation of a second solution. A third method of preparing the liquid formulation is similar to the above-described first method except that the SAE-CD is added directly to an aqueous second solution containing the sertraline without formation of the first solution. A fourth method of preparing the liquid formulation comprises the steps of adding an aqueous solution comprising sertraline to a powdered or particulate SAE-CD and mixing the solution until the SAE-CD has dissolved. A fifth method of preparing the liquid formation comprises the steps of adding the sertraline directly to the powdered or particulate SAE-CD and then adding an aqueous solution and mixing until the SAE-CD and sertraline has dissolved. A sixth method for preparing the liquid formation comprises the steps of heating either the first solution or heating the second solution, or heating a combination thereof of any solutions described in the above methods followed by the step of cooling the respectively heated solution. A seventh method for preparing the liquid formation comprises the step of adjusting the pH of either the first solution or adjusting the pH of the second solution or adjusting the pH of a combination of either solutions described in any of the above methods. An eighth method comprises the steps of creating the liquid formulation by any of the above-described methods followed by the step of isolating a solid material by lyophilization, spray-drying, spray freeze-drying, vacuum-drying, antisolvent precipitation or a process utilizing a supercritical or near supercritical fluid. Any of the above solutions can contain other pharmaceutical excipients or ingredients as described herein.

Specific embodiments of the method of preparing the liquid formulation include those wherein the method further comprises the step of: 1) filtering the formulation through a filtration medium wherein the pore size is about 5 μm or smaller; 2) sterilizing the liquid formulation by irradiation; 3) sterilizing the liquid formulation by treatment with ethylene oxide; 4) isolating a sterile powder from the sterilized liquid formulation; 5) purging the liquid with an inert gas to reduce the amount of dissolved oxygen in the liquid; and/or 6) one or more of the solutions used to prepare the liquid formulation is heated.

The liquid formulation of the invention can be provided in a kit. The kit will comprise a first pharmaceutical composition comprising an SAE-CD and a second pharmaceutical composition comprising sertraline. The first and second formulations can be mixed and formulated as a liquid dosage form prior to administration to a subject. Either one or both of the first and second pharmaceutical compositions can comprise additional pharmaceutical excipients. The kit is available in various forms.

In a first kit, the first and second pharmaceutical compositions are provided in separate containers or separate chambers of a container having two or more chambers. The first and second pharmaceutical compositions may be independently provided in either solid or powder or liquid form. For example, the SAE-CD can be provided in a reconstitutable powder form and sertraline can be provided in powdered form. According to one embodiment, the kit would further comprise a pharmaceutically acceptable liquid carrier used to suspend and dissolve the first and/or second pharmaceutical compositions. Alternatively, a liquid carrier is independently included with the first and/or second pharmaceutical composition. The liquid carrier, however, can also be provided in a container or chamber separate from the first and second pharmaceutical compositions. As above, the first pharmaceutical composition, the second pharmaceutical composition and the liquid carrier can independently comprise a preservative, an antioxidant, a buffering agent, an acidifying agent, an electrolyte, another therapeutic agent, an alkalizing agent, an antimicrobial agent, an antifungal agent, a solubility enhancing agent, a viscosity modifying agent, a flavoring agent, a sweetening agent or a combination thereof.

Specific embodiments of the kit include those wherein: 1) the first and second pharmaceutical compositions are contained in separate containers or separate chambers of a container having two or more chambers; 2) the kit further comprises a separate pharmaceutically acceptable liquid carrier; 3) a liquid carrier is included with the first and/or second pharmaceutical composition; 4) containers for the pharmaceutical compositions are independently selected at each occurrence from an evacuated container, bag, pouch, vial, bottle, or any pharmaceutically acceptable device known to those skilled in the art for the delivery of liquid formulations; 5) the first pharmaceutical composition and/or second pharmaceutical composition and/or liquid carrier further comprises an antioxidant, a buffering agent, an acidifying agent, a solubilizing agent, a complexation enhancing agent, lyophilizing aids (for example, bulking agents or stabilizing agents), an electrolyte, another therapeutic agent, an alkalizing agent, an antimicrobial agent, an antifungal agent, a viscosity modifying agent, a flavoring agent, a sweetening agent or a combination thereof; 6) the kit is provided chilled; 8) the liquid carrier and/or chamber has been purged with a pharmaceutically acceptable inert gas to remove substantially all of the oxygen dissolved in the liquid carrier; 9) the chambers are substantially free from oxygen; 10) the liquid carrier further comprises a buffering agent capable of maintaining a pH of about 2-7; 11) the chambers and solutions are sterile.

The term “unit dosage form” is used herein to mean a single or multiple dose form containing a quantity of the active ingredient and the diluent or carrier, said quantity being such that one or more predetermined units are normally required for a single therapeutic administration. In the case of multiple dose forms, such as liquid-filled bottles, said predetermined unit will be one fraction such as a half or quarter of the multiple dose form. It will be understood that the specific dose level for any patient will depend upon a variety of factors including the indication being treated, therapeutic agent employed, the activity of therapeutic agent, severity of the indication, patient health, age, sex, weight, diet, and pharmacological response, the specific dosage form employed and other such factors.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is taken to mean warm blooded animals such as mammals, for example, cats, dogs, mice, guinea pigs, horses, bovine cows, sheep, and humans.

The liquid formulation of the invention will comprise an effective amount of sertraline. By the term “effective amount”, it is understood that a therapeutically effective amount is contemplated. A therapeutically effective amount is the amount or quantity of sertraline that is sufficient to elicit the required or desired therapeutic response, or in other words, the amount that is sufficient to elicit an appreciable biological response when administered to a subject.

The typical daily doses for sertraline, expressed as the free base, range from 50-200 mg, increasing in 50 mg increments. A titration dose of 25 mg/day during the initial phase of therapy may be warranted in some indications. Since the present formulations are substantially bioequivalent to the ZOLOFT® oral concentrate formulation, they can be administered as directed in the package insert for the ZOLOFT® oral concentrate formulation. The Physician's Desk Reference 56^{th} ed. (pp. 2751-2756; Eds. Lori Murray, Gwynned L. Kelly; Medical Economics Company, Inc., Montvale, N.J. 07645-1742, 2002), the relevant text of which is hereby incorporated by reference, discloses the package insert for ZOLOFT®, and particularly the dosage and administration for the oral concentrate solution.

In view of the above description and the examples below, one of ordinary skill in the art will be able to practice the invention as claimed without undue experimentation. The foregoing will be better understood with reference to the following examples that detail certain procedures for the preparation of formulations according to the present invention. All references made to these examples are for the purposes of illustration. The following examples should not be considered exhaustive, but merely illustrative of only a few of the many embodiments contemplated by the present invention.

### EXAMPLE 1

The phase solubility curves for sertraline with SAE-CD, HP-β-CD and γ-CD were determined according to procedures well known in the art (Higuchi et al. in Phase Solubility Techniques, in Advances in Analytical Chemistry and Instrumentation (Ed. C. N. Reilly, John Wiley & Sons Inc., Vol. 4 (1965), pg. 117-212) the relevant disclosure of which is hereby incorporated by reference). The results are depicted in FIG. 1.

### EXAMPLE 2

A sweetened, unflavored aqueous solution of sertraline hydrochloride was prepared at native pH. The formulation comprised Captisol® (SBE7-β-CD) (15% wt./vol.) and polymorph II of sertraline hydrochloride. The amounts used are specified in the table below.

| Ingredients | Amount | |
|---|---|---|
| Sertraline hydrochloride | 1.0 | g (equivalent to |
| | 0.894 | g sertraline) |
| SBE7-β-CD | 7.5 | g (anhydrous |
| | | basis) |
| Xylitol | 15 | g |
| Sodium saccharin | 0.05 | g |

| | |
|---|---|
| Water | qs to 50 mL |

The following procedure was used to prepare the formulation. Seven and one half grams of SBE7-β-CD were added to approximately 30 mL water and dissolved with mixing at room temperature. The following ingredients were then individually added and dissolved in the solution with stirring; 1.0 g sertraline hydrochloride and 0.50 g sodium saccharin. Fifteen grams of xylitol were added along with an additional 10 mL water with continued stirring. The solution was then heated to about 50 degrees C. to facilitate the dissolution of the xylitol. The solution was allowed to cool to room temperature (22-25 degrees C.) then was brought to a final volume of 50 mL with water. The solution had a pH of 5.45.

### EXAMPLE 3

A sweetened, unflavored aqueous solution of sertraline hydrochloride was prepared at native pH. The solution contained Captisol® (17% wt./vol.) and sertraline hydrochloride (polymorph II at a concentration of 20 mg/mL). The following ingredients were used in the amounts indicated.

| Ingredients | Amount | |
|---|---|---|
| Sertraline hydrochloride | 1.119 | g |
| SBE7-β-CD | 8.5 | g (anhydrous |
| | | basis) |
| Xylitol | 15 | g |
| Sodium saccharin | 0.05 | g |

| | |
|---|---|
| Water | qs to 50 mL |

The liquid formulation was prepared as follows. 8.5 grams of SBE7-β-CD were added to approximately 30 mL water and dissolved with mixing at room temperature. The following ingredients were then individually added and dissolved in the solution with stirring; 1.119 g sertraline hydrochloride and 0.50 g sodium saccharin. Fifteen grams of xylitol were added along with an additional 10 mL water with continued stirring. The solution was then heated to about 50 degrees C. to facilitate the dissolution of the xylitol. The solution was allowed to cool to room temperature (22-25 degrees C.) then was brought to a final volume of 50 mL with water. The solution had a pH of 5.35.

### EXAMPLE 4

A sweetened, unflavored aqueous solution of sertraline hydrochloride was prepared. The liquid formulation contained benzoic acid as an antimicrobial preservative. The formulation contained SBE7-β-CD (17% wt./vol.), xylitol and sorbitol. The following ingredients were used in the amounts indicated.

| Ingredients | Amount | |
|---|---|---|
| Sertraline hydrochloride | 1.19 | g |
| SBE7-β-CD | 8.5 | g |
| Xylitol | 15 | g |
| Sodium saccharin | 0.05 | g |
| Citric acid | 0.150 | g |
| Benzoic acid | 0.05 | g |
| Glycerin | 5 | g |
| Sorbitol | 5 | g |

| | |
|---|---|
| Sodium hydroxide (1N) | as need for pH 4.0 |
| Water | qs to 50 mL |

The formulation was prepared as follows. Eight and one-half grams of SBE7-β-CD were added to approximately 20 mL water dissolved with stirring. The following ingredients were individually added and dissolved in the solution with mixing; 0.05 g benzoic acid, 1.119 g sertraline hydrochloride, 0.05 g sodium saccharin and 0.15 g citric acid. Glycerin (5 g), xylitol (15 g), and sorbitol (5 g) were added to the solution and dissolved with continued stirring. The solution was heated to approximately 50 degrees C. to facilitate dissolution. The solution was allowed to cool to room temperature (22-25 degrees C.) then the pH was adjusted to 4.0 with 1N sodium hydroxide. The solution was brought to final volume of 50 mL with water, mixed well and filtered through a 5 micron pore size filter.

### EXAMPLE 5

A sweetened, unflavored aqueous solution of sertraline hydrochloride was prepared. The formulation contained SBECD (15% wt./vol.). The procedure was identical to that of Example 4 except 7.5 g of SBECD, instead of 8.5 g, were used.

### EXAMPLE 6

A sweetened, unflavored aqueous solution of sertraline hydrochloride was prepared using polymorph I of sertraline hydrochloride. The following ingredients were. used in the amounts indicated.

| Ingredients | Amount | |
|---|---|---|
| Sertraline hydrochloride | 1.12 | g |
| SBE7-β-CD | 8.5 | g |
| Xylitol | 22.5 | g |
| Sodium saccharin | 0.05 | g |
| Citric acid | 0.15 | g |
| Benzoic acid | 0.05 | g |
| Glycerin | 5 | g |

| | |
|---|---|
| Sodium hydroxide (1N) | as need for pH 4.0 |
| Water | qs to 50 mL |

The following procedure was used. Eight and one-half grams of SBE7-β-CD were added to approximately 18 mL water and dissolved with the aid of an overhead high-speed mixer. Benzoic acid (0.05 g) was added and dissolved then the sertraline hydrochloride (polymorph II, 1.12 g) was added. High speed mixing was continued for 3.5 hours until the sertraline was dissolved. The following ingredients were individually added and dissolved in the solution; 0.05 g sodium saccharin and 0.15 g citric acid, glycerin (5 g) and xylitol (22.5 g) with continued stirring. The solution was heated to about 50 degrees C. to facilitate the dissolution of the xylitol. The solution was allowed to cool to room temperature (22-25 degrees C.) then the pH was adjusted to 4.0 with 1N sodium hydroxide. The solution was brought to final volume of 50 mL with water and mixed well.

### EXAMPLE 7

A sweetened, unflavored aqueous solution of sertraline hydrochloride was prepared using polymorph I of sertraline hydrochloride. The following ingredients were used in the amounts shown.

| Ingredients | Amount | |
|---|---|---|
| Sertraline hydrochloride | 2.238 | g |
| SBE7-β-CD | 17.0 | g |
| Xylitol | 40 | g |
| Sodium saccharin | 0.10 | g |
| Citric acid | 0.30 | g |
| Glycerin | 10 | g |

| | |
|---|---|
| Sodium hydroxide (1N) | as need for pH 4.0 |
| Water | qs to 100 mL |

The formulations were prepared as follows. Approximately 40 mL of water were heated to 55 degrees C. Seventeen grams of SBE7-β-CD were added and dissolved with mixing. Sertraline hydrochloride (polymorph I, 2.238 g) was added and dissolved with continuous stirring. Dissolution time was approximately 45 minutes. The following ingredients were individually added and dissolved in the solution with mixing; 0.10 g sodium saccharin and 0.30 g citric acid, glycerin (10 g) and xylitol (40 g) with continued stirring. The solution was allowed to cool to room temperature (22-25 degrees C.) then brought to a final volume of 100 mL with water and mixed well. The resultant pH was 4.08. The solution was passed through a 5 micron nylon filter.

### EXAMPLE 8

The stability of five sertraline liquid formulations was determined after exposure to stress by either fluorescent light or ultraviolet light. The formulations included the marketed non-aqueous ZOLOFT Oral Concentrate and four formulations containing equimolar amounts of different cyclodextrins or cyclodextrin derivatives and sertraline. All formulations contained 22.5 mg/mL sertraline HCl, equivalent to 20 mg/mL sertraline free base and cyclodextrin at 0.078M. The cyclodextrin formulations were prepared by dissolving the appropriate amount of cyclodextrin in ˜9 mL HPLC grade water, adding the sertraline, and mixing until all the sertraline was dissolved. The solutions were brought to a final volume of 10 mL with water then passed through a 0.22 micron Millex-GV Durapore filter. Each of the solutions were analyzed for content of sertraline and presence of degradants by HPLC. Aliquots (1.5 mL) of each solution A-E were placed in 1 dram glass vials with Teflon-lined screw-caps and stored exposed to high intensity fluorescent light (˜25 cm from a bank of Sylvania Cool White 15 watt lamps) for 15 days. Aliquots (1.5 mL) of each solution A-E were also placed in 10 mL glass beakers, covered tightly with a thin plastic wrap and centered ˜10 cm beneath 2-20 watt SilverLite XL F20W Blacklight Blue (ultraviolet) lamps for 15 days. At the end of the 15 day storage period, each of the samples were assayed by the HPLC method and the amounts of each of the main degradants calculated as a percentage of sertraline peak area appearing in the chromatogram.

Formulations tested:

Formulation A: Sertraline plus 2-hydroxypropyl-β-cyclodextrin (DS=6.7)

Formulation B: Sertraline plus Sulfobutylether-β-cyclodextrin (DS=5.5)

Formulation C: Sertraline plus Sulfobutylether-β-cyclodextrin (DS=6.7)

Formulation D: Sertraline plus gamma-CD

Formulation E: ZOLOFT® Oral Concentrate

The results are given in the table below, for each formulation and quantitated for each major degradant found on the chromatogram, and also summed as the total amounts of degradants. The amount of each degradant formed upon storage is reported as the ratio of its peak area to the peak area of sertraline. The peak area of sertraline did not change significantly over the course of the study. The degradants are identified by their chromatographic retention times, tr, on an HPLC system using a Phenomenex Luna 5 μm, 250×4.6 mm CN column and a mobile phase containing 50% 0.05M monosodium phosphate pH 6 and 50% acetonitrile flowing at 1.0 mL/min. Detection was by uv absorption at 220 nm. Sertraline retention time was ˜21 minutes on this analytical system.

Peak area as a percentage of sertraline peak area

| | | |
|---|---|---|
| Formulation | Fluorescent Light | Ultraviolet Light |

Degradant peak at tr = 2.8 min

| | | |
|---|---|---|
| A | 0.416 | 0.395 |
| B | 0.0107 | 0.012 |
| C | 0.0069 | 0.0088 |
| D | 0.548 | 0.644 |
| E | 0.048 | 0.0891 |

Degradant peak at tr = 6.1 min

| | | |
|---|---|---|
| A | 0.161 | 0.152 |
| B | 0.112 | 0.066 |
| C | 0.0066 | 0.054 |
| D | 0.535 | 0.215 |
| E | 0.234 | 0.262 |

Degradant peak at tr = 11.0 min

| | | |
|---|---|---|
| A | 0.0080 | 0.0029 |
| B | 0.0013 | 0.0009 |
| C | 0.0006 | 0.0006 |
| D | 0.099 | 0.153 |
| E | 0.230 | 0.318 |

Degradant peak at tr = 13.9 min

| | | |
|---|---|---|
| A | 0.107 | 0.135 |
| B | 0.0228 | 0.025 |
| C | 0.0060 | 0.011 |
| D | 11.5 | 0.843 |
| E | 3.08 | 0.026 |

Degradant peak at tr = 17.1 min

| | | |
|---|---|---|
| A | 0.132 | 0.176 |
| B | 0.104 | 0.120 |
| C | 0.099 | 0 |
| D | 0.182 | 0.375 |
| E | 0.310 | 1.62 |

Total Degradants

| | | |
|---|---|---|
| A | 0.9527 | 0.9034 |
| B | 0.2727 | 0.2237 |
| C | 0.3214 | 0.0728 |
| D | 13.1124 | 2.5536 |
| E | 5.0022 | 4.1485 |

### EXAMPLE 9

A clinical study was conducted in 12 adult subjects of mixed gender comparing the pharmacokinetics of sertraline after dosing with a formulation of the invention, prepared according to Example 7, or as ZOLOFT Oral Concentrate. The study was designed such that each subject received each formulation in a crossover manner with a 14 day washout between dosings. The formulation of the invention was dosed directly to the subjects as a 5 mL aliquot of the liquid (100 mg sertraline). The subjects then consumed 120 mL lemon/lime soda and 120 mL water. The ZOLOFT Oral Concentrate dose of 5 mL (100 mg sertraline) was diluted in 120 mL lemon/lime soda then administered to the subjects. Each subject then received 120 mL water.

Blood samples were withdrawn from each subject over 72 hours after dosing of each formulation and analyzed for sertraline content. Pharmacokinetic parameters were then calculated from the sertraline blood level-time profile. The results, shown in the table below, indicate that the two formulations give equivalent pharmacokinetic parameters.

| | | | | ZOLOFT Oral | |
|---|---|---|---|---|---|
| Pharmacokinetic | | Cyclodextrin/Sertraline | | Concentrate Arithmetic | |
| Parameter | Parameter Definition | Arithmetic Mean (SD) | | Mean (SD) | |
| Cmax (ng/mL) | Maximal plasma concentration | 34.9 | (15.4) | 38.0 | (16.2) |
| Tmax (hr) | Time of maximal concentration | 5.09 | (1.87) | 4.73 | (1.62) |
| AUC(0-72) (ng*hr/mL) | Area under the plasma | 843.6 | (337.3) | 917.0 | (548.9) |
| | concentration/time profile from | | | | |
| | time = 0 to 72 hours | | | | |
| AUC(0-inf) (ng*hr/mL) | Area under the plasma | 1030 | (491.6) | 1112 | (734.2) |
| | concentration/time profile from | | | | |
| | time = 0 to infinity | | | | |
| T½ (hr) | Half life for drug elimination | 30.1 | (10.1) | 28.9 | (5.2) |
| Kel (1/hr) | Terminal phase elimination rate | 0.0247 | (0.00572) | 0.0247 | (0.00462) |
| | constant | | | | |

### EXAMPLE 10

Method 1. Volunteers in the study in Example 9 rated the taste of each of the formulations immediately after ingestion, on a scale of 1 to 5 using the following guide; 1=very bad, 2=bad, 3=neither good nor bad, 4=good, 5=very good. The cyclodextrin formulation of the invention (mean rating of 3.91+/±0.83 s.d.) had a better taste than the ZOLOFT® Oral Concentrate (mean of 2.64+/−1.03 s.d.). The difference was significant at p<0.05.

Method 2. Two aqueous formulations were prepared containing 20 mg/mL sertraline: one with sulfobutylether-β-CD (SBECD, degree of substitution (DS)=6.7) and one with 2-hydroxypropyl-β-CD (HPCD, DS=6.7). Each solution was prepared by dissolving the cyclodextrin in water, then adding the sertraline (as 22.4 mg/mL of the HCl salt) with stirring until it was dissolved. The SBECD formulation was labeled A and the HPCD formulation was labeled B. Eight volunteers, blinded to the identity of the formulations, tasted each of the formulations in random order with a 1 hour wait between formulations. The volunteers placed 0.5 mL of each formulation in their mouth, swished the solution for up to 15 seconds then spat out the solution. They then rated the taste of the solution on a scale of 1 to 5 (1-very bad, 2-bad, 3-neither good nor bad, 4-good, and 5-very good). Formulation A received an average rating of 2.6±0.5 and formulation B received an average rating of 1.8±0.6 indicating the SBECD formulation tasted better than the HPCD formulation.

Method 3. Aqueous solutions were prepared containing 22.4 mg/mL sertraline HCL (equivalent to 20 mg/mL sertraline) and 0.069M of various cyclodextrins. The cyclodextrins used were:

I-gamma-cyclodextrin

II-2-hydroxypropyl-β-CD

III-sulfobutylether-β-cyclodextrin, calcium salt (Ca-SAE-CD)

IV-sulfobutylether-β-cyclodextrin, ammonium salt (NH4-SAE-CD)

V-sulfobutylether-β-cyclodextrin, sodium salt (Na-SAE-CD)

A ½ mL aliquot of each solution was tasted in random order by three volunteers, blinded to the selection of the cyclodextrin. The volunteers recorded their observations.

### EXAMPLE 11

A sweetened, flavored aqueous solution of sertraline hydrochloride is prepared using polymorph I of sertraline hydrochloride. The following ingredients are used in the amounts shown.

| Ingredients | Amount | |
|---|---|---|
| Sertraline hydrochloride | 2.238 | g |
| SBE4-β-CD | 14.0 | g |
| Xylitol | 40 | g |
| Sodium saccharin | 0.10 | g |
| Citric acid | 0.30 | g |
| Glycerin | 10 | g |
| Watermelon flavor | 1.5 | g |

| | |
|---|---|
| Sodium hydroxide (1N) | as need for pH 4.0 |
| Water | qs to 100 mL |

The formulation is prepared by heating approximately 40 mL of water to 55 degrees C. Fourteen grams of SBE4-β-CD are added and dissolved with mixing. Sertraline hydrochloride (polymorph I, 2.238 g) is added and dissolved with continuous stirring. The following ingredients are individually added and dissolved in the solution with mixing; 0.10 g sodium saccharin and 0.30 g citric acid, glycerin (10 g) and xylitol (40 g) with continued stirring. The solution is allowed to cool to room temperature (22-25 degrees C.). Watermelon flavor (1.5 g) is added to the solution which is then brought to a final volume of 100 mL with water and mixed well.

### EXAMPLE 12

A sweetened, unflavored aqueous solution of sertraline hydrochloride was prepared which contained benzoic acid as an antimicrobial preservative. The following ingredients were used in the amounts indicated.

| Ingredients | Amount | |
|---|---|---|
| Sertraline hydrochloride | 5.595 | g |
| SBE7-β-CD | 42.5 | g |
| Xylitol | 100 | g |
| Sodium saccharin | 0.25 | g |
| Citric acid | 0.75 | g |
| Benzoic acid | 0.25 | g |
| Glycerin | 25 | g |

| | |
|---|---|
| Sodium hydroxide (1N) | as need for pH 4.0 |
| Water | qs to 250 mL |

The formulation was prepared as follows. Approximately 100 mL water was heated under mild agitation to 55-60 degrees C. then 42.5 grams of SBE7-β-CD were added and dissolved with continued mixing. The following ingredients were individually added and dissolved in the solution; 0.25 g benzoic acid, 5.595 g sertraline hydrochloride, 0.25 g sodium saccharin and 0.75 g citric acid. Glycerin (25 g) and xylitol (100 g) were added to the solution and dissolved with continued stirring. The solution was allowed to cool to room temperature (22-25 degrees C.) then the pH was adjusted to 4.0 with 1N sodium hydroxide. The solution was brought to final volume of 250 mL with water, mixed well and filtered through a 5 micron pore size filter.

### EXAMPLE 13

A sweetened, unflavored aqueous solution of sertraline hydrochloride was prepared which contained sorbic acid as an antimicrobial preservative. The following ingredients were used in the amounts indicated.

| Ingredients | Amount | |
|---|---|---|
| Sertraline hydrochloride | 5.595 | g |
| SBE7-β-CD | 42.5 | g |
| Xylitol | 100 | g |
| Sodium saccharin | 0.25 | g |
| Citric acid | 0.75 | g |
| Sorbic acid | 0.50 | g |
| Glycerin | 25 | g |

| | |
|---|---|
| Sodium hydroxide (1N) | as need for pH 4.0 |
| Water | qs to 250 mL |

The formulation was prepared as follows. A solution was prepared as in Example 12 except 0.5 g of sorbic acid was used as the preservative in place of the 0.25 g benzoic acid.

### EXAMPLE 14

The microbial growth retarding capability of formulations of Examples 12 and 13 were tested according to the procedures outlined in the United States Pharmacopeia 27, 2004 (USP), <51> Antimicrobial Effectiveness Testing, and the European Pharmacopoeia 4^{th} Edition 2003 (EP). The formulations were evaluated in duplicate employing a liquid-to-liquid matrix against five test organisms, then quantitated using membrane filtration. Approximately 1×10⁵ to 1×10⁶ colony forming units (CFU) per mL of five standard organisms recommended by the USP for preservative efficacy tests were inoculated in each formulation. These five organisms are identified as Staphylococcus aureus (ATCC 6538), Pseudomonas aeruginosa (ATCC 9027), Escherichia coli (ATCC 8739), Aspergillus niger (ATCC 16404) and Candida albicans (ATCC 10231).

The antimicrobial activity of the two formulations is illustrated in the following table as log reduction in microbial count from the count at zero time.

| | Decrease in viable survivors (log₁₀ CFU/mL) | | | | |
|---|---|---|---|---|---|
| | A. niger | C. albicans | E. coli | P. aeruginosa | S. aureus |
| Formulation of Example 11 | | | | | |
| 14 day | 1.93 | 1.06 | >4.76 | >4.80 | >4.85 |
| 28 day | >4.37 | >4.79 | >4.76 | >4.80 | >4.85 |
| USP Pass/Fail | Pass | Pass | Pass | Pass | Pass |
| EP Pass/Fail | Pass | Pass | Pass | Pass | Pass |
| Formulation of Example 12 | | | | | |
| 14 day | >4.37 | >4.79 | >4.76 | >4.80 | >4.85 |
| 28 day | >4.37 | >4.79 | >4.76 | >4.80 | >4.85 |
| USP Pass/Fail | Pass | Pass | Pass | Pass | Pass |
| EP Pass/Fail | Pass | Pass | Pass | Pass | Pass |
| USP Criteria | | | | | |
| 14 day | no increase | no increase | 1 | 1 | 1 |
| 28 day | no increase | no increase | no increase | no increase | no increase |
| EP Criteria | | | | | |
| 14 day | 1 | 1 | 3 | 3 | 3 |
| 28 day | no increase | no increase | no increase | no increase | no increase |

### EXAMPLE 15

The dilutability of the present formulations as compared to that of the commercial ZOLOFT® formulation were compared as follows. A formulation of the invention was prepared according to Example 7. Five milliliter aliquots of the formulation or of ZOLOFT Oral Concentrate, equivalent to 100 mg sertraline, were added to 120 mL of each of several diluents. The resulting solutions were visually checked at 5 minutes and 30 minutes after preparation for the appearance of physical changes such as changes in color or the formation of a precipitate or other immiscible phase.

### EXAMPLE 16

A sweetened, flavored aqueous solution of sertraline hydrochloride is prepared using polymorph I of sertraline hydrochloride and the sulfobutyl ether-gamma-cyclodextrin with a degree of substitution (DS) of ˜5. The following ingredients are used in the amounts indicated.

| Ingredients | Amount | |
|---|---|---|
| Sertraline hydrochloride | 1.12 | g |
| SBE5-γ-CD | 8.12 | g |
| Xylitol | 22.5 | g |
| Sodium saccharin | 0.05 | g |
| Citric acid | 0.15 | g |
| Benzoic acid | 0.05 | g |
| Glycerin | 5 | g |
| Raspberry flavor | 0.75 | g |

| | |
|---|---|
| Sodium hydroxide (1N) | as need for pH 4.0 |
| Water | qs to 50 mL |

The following procedure was used. Sulfobutyl ether-gamma-cyclodextrin (DS=5), 8.12 grams, are added to approximately 18 mL water and dissolved with the aid of an overhead high-speed mixer. Benzoic acid (0.05 g) is added and dissolved then the sertraline hydrochloride (polymorph I, 1.12 g) is added. High speed mixing is continued for 3.5 hours until the sertraline is dissolved. The following ingredients are individually added and dissolved in the solution; 0.05 g sodium saccharin and 0.15 g citric acid, glycerin (5 g) and xylitol (22.5 g) with continued stirring. The solution is heated to about 50 degrees C. to facilitate the dissolution of the xylitol. The solution is allowed to cool to room temperature (22-25 degrees C.), 0.75 grams of raspberry flavor were added. The pH is adjusted to 4.0 with 1N sodium hydroxide and the solution is brought to final volume of 25 mL with water and mixed well.

### EXAMPLE 16

Aqueous 0.01M buffers were prepared at pH values of 1 (HCl), 3 and 5 (citric acid/sodium citrate), and 7 (monobasic potassium phosphate) and used to prepare solutions containing 0, 10% or 20% sulfobutylether-β-cyclodextrin (DS=6.7). Excess sertraline HCl was added to each of the solutions and allowed to mix for three days. The solutions were centrifuged and the supernatants analyzed for sertraline content and final pH.

### EXAMPLE 17

A sweetened and flavored aqueous solution of sertraline hydrochloride (equivalent to 10 mg/mL sertraline) was prepared which contained benzoic acid as an antimicrobial preservative and a citric acid/sodium citrate buffer. The following ingredients were used in the amounts indicated.

| Ingredients | Amount | |
|---|---|---|
| SBE7-β-CD (corrected for water content) | 11 | g |
| Sertraline hydrochloride | 1.12 | g* |
| Benzoic acid | 0.15 | g |
| Citric acid, monohydrate | 0.274 | g |
| Sodium citrate, dihydrate | 0.317 | g |
| Glycerin | 10 | g |
| Xylitol | 20 | g |
| Strawberry flavor | 0.17 | g |

| | |
|---|---|
| Sodium hydroxide (1N) | as need for pH 4.0 |
| Water | qs to 100 mL |

*equivalent to 10 mg/mL sertraline

The formulation was prepared as follows. Approximately 50 mL water was heated under mild agitation to 55-60 degrees C. then 11 grams of SBE7-β-CD were added and dissolved with continued mixing. The following ingredients were individually added and dissolved in the solution; 1.12 g sertraline hydrochloride, 0.15 g benzoic acid, 0.274 g citric acid monohydrate, and 0.317 g sodium citrate dihydrate. Glycerin (10 g) and xylitol (20 g) were added to the solution and dissolved with continued stirring. The solution was allowed to cool to room temperature (22-25 degrees C.) then the pH was adjusted to 4.0 with 1N sodium hydroxide. Strawberry flavor (0.171 g) was added to the solution and stirred until dissolved. The solution was brought to final volume of 100 mL with water, mixed well and filtered through a 5 micron pore size filter.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except by the appended claims. All of the embodiments disclosed and claimed herein can be made and executed without undue experimentation in light of the present disclosure.

1. A taste-masked aqueous oral liquid formulation comprising sertraline, SAE-CD, an aqueous liquid carrier, wherein the molar ratio of SAE-CD to sertraline is at least 0.95 and the formulation possesses improved taste over an otherwise similar aqueous formulation excluding SAE-CD.

2. The formulation of claim 1, wherein the formulation has improved taste as compared to an otherwise similar aqueous formulation comprising HP-β-CD present in place of SAE-CD in equimolar amounts therewith.

3. The formulation of claim 1, wherein the sertraline is provided as a pharmaceutically acceptable salt.

4. The formulation of claim 1, wherein the SAE-CD is a compound, or mixture of compounds, of the Formula 1 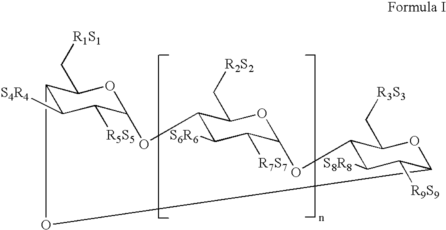
wherein:
n is 4, 5, or 6;
R₁, R₂, R₃, R₄, R₅, R₆, R₇, R₈, and R₉ are each, independently, —O— or a —O-(C₂-C₆alkylene)-SO₃⁻ group, wherein at least one of R₁ and R₂ is independently a —O-(C₂-C₆ alkylene)-SO₃⁻ group; and
S₁, S₂, S₃, S₄, S₅, S₆, S₇, S₈, and S₉ are each, independently, a pharmaceutically acceptable cation.

5. The formulation of claim 4, wherein the compound of Formula 1 has a degree of substitution of about 4 or 7.

6. The formulation of claim 1 further comprising a acidifying agent, alkalizing agent, antifungal agent, antimicrobial agent, antioxidant, another therapeutic agent, buffering agent, bulking agent, complexation enhancing agent, cryoprotectant, density modifier, electrolyte, flavor, fragrance, lyophilizing aid, preservative, plasticizer, solubility-enhancing agent, stabilizing agent, sweetener, surface tension modifier, volatility modifier, viscosity modifier, or a combination thereof.

7. The formulation of claim 6, wherein the buffering agent is an organic or inorganic acid, organic or inorganic base, or salt thereof.

8. The formulation of claim 7, wherein the buffering agent is selected from the group consisting of acetic acid, citric acid, phosphoric acid, boric acid, or a salt thereof.

9. The formulation of claim 6, wherein the formulation comprises sertraline, SBE7-β-CD, xylitol, citric acid, sodium citrate, glycerin, benzoic acid, a flavor, and water, and the pH of the formulation is in the range of about 2-7.

10. The formulation of claim 1 further comprising a flavor.

11. The formulation of claim 1, wherein the formulation possesses a greater photochemical stability to fluorescent and/or ultraviolet light than does an non-aqueous type formulation comprising glycerin, ethanol, menthol, butylated hydroxytoluene (BHT) and a comparable amount of sertraline.

12. The formulation of claim 1, wherein sertraline is present at a concentration of about 1-110 mg/mL.

13. The formulation of claim 1, wherein the formulation possesses a more acceptable taste than does a non-aqueous type formulation comprising glycerin, ethanol, menthol, butylated hydroxytoluene (BHT) and a comparable amount of sertraline.

14. The formulation of claim 6, wherein the preservative is sorbic acid or benzoic acid.

15. The formulation of claim 1, wherein the formulation provides substantially equivalent pharmacokinetics as an non-aqueous type formulation comprising glycerin, ethanol, menthol, butylated hydroxytoluene (BHT) and a comparable amount of sertraline.

16. The formulation of claim 1, wherein SAE-CD is present at a concentration of about 5 to 700 mg/mL.

17. The formulation of claim 1, wherein SAE-CD is a compound, or mixture of compounds, of the formula SAEx-R-CD (Formula 2), wherein
a. SAE is selected from the group consisting of sulfomethyl ether, sulfoethyl ether, sulfopropyl ether, sulfobutyl ether, sulfopentyl ether, and sulfohexyl ether; and
b. x is in the range of about 1-18, 1-21, or 1-24, when R is α, β or γ, respectively.

18. The formulation of claim 1, wherein the liquid formulation has been prepared by reconstitution of a reconstitutable solid comprising at least SAE-CD and sertraline with an aqueous solution.

19. The formulation of claim 1, wherein the formulation is a ready-to-use formulation not requiring dilution prior to oral administration to a subject.

20. The formulation of claim 1, wherein the formulation is dilutable with an aqueous diluent without significant precipitation of the sertraline.

21. The formulation of claim 20, wherein the diluent is selected from the group consisting of lemon/lime soda, ginger ale soda, cola soda, orange juice, or apple juice.

22. A method for preparing a taste-masked aqueous liquid oral formulation from a reconstitutable solid, the method comprising the steps of:
a. providing a reconstitutable solid comprising sertraline, SAE-CD and optionally at least one other pharmaceutical excipient, wherein the solid is reconstitutable with an aqueous liquid, and the molar ratio of SAE-CD to sertraline is at least about 0.95; and
b. reconstituting the solid with a sufficient amount of aqueous liquid carrier sufficient to at least suspend the reconstitutable solid, thereby forming the taste-masked aqueous liquid oral formulation.

23. The method of claim 22, wherein the reconstitutable solid comprises an admixture of sertraline, SAE-CD and optionally one or more excipients, wherein a major portion of the sertraline is not complexed with the SAE-CD.

24. The method of claim 22, wherein the reconstitutable solid comprises a preformed complex of sertraline and SAE-CD and optionally one or more excipients, wherein a major portion of the sertraline is complexed with the SAE-CD.

25. The method of claim 22, wherein after reconstitution, the liquid formulation is ready for oral administration to a subject without requiring further dilution.

26. The method of claim 22, wherein the liquid formulation is a suspension.

27. The method of claim 22, wherein the amount of liquid carrier added is sufficient to render the liquid formulation clear.

28. The method of claim 22, wherein the formulation is a concentrate comprising at least 1 mg of sertraline in a volume of 1 ml.

29. The method of claim 22, wherein

30. A method of treating depression comprising orally administering to a subject in need thereof a formulation according to claim 1.

31. A method of treating or preventing diseases or conditions that are caused by disorders of the serotonergic system, the method comprising the step of orally administering to a subject in need thereof a formulation according to claim 1.

32. The method of claim 31, wherein the disease or condition is selected from the group consisting of depression, anorexia, chemical dependencies, anxiety-related disorder, panic disorder, obsessive-compulsive disorder, generalized anxiety disorder, phobia, post traumatic stress disorder, avoidant personality disorder, premature ejaculation, cancer and post myocardial infarction.

33. A method of orally administering to a subject sertraline in a taste-masked aqueous liquid formulation, the method comprising the steps of:
a. providing an aqueous liquid comprising SAE-CD, sertraline and an aqueous carrier, wherein the molar ratio of SAE-CD to sertraline is at least about 0.95;
b. diluting the liquid with a pharmaceutically acceptable aqueous diluent to form a ready-to-administer taste-masked aqueous liquid formulation; and
c. orally administering to a subject at least one unit dose of the ready-to-administer formulation.

34. The method of claim 33, wherein the sulfoalkyl ether cyclodextrin is a compound or mixture of compounds of the Formula 1.

35. A clear taste-masked aqueous oral liquid formulation comprising a therapeutically effective amount of sertraline, SAE-CD, an aqueous liquid carrier, one or more sweeteners, and, optionally, one or more pharmaceutically acceptable excipients, wherein the molar ratio of SAE-CD to sertraline is at least 0.95.

36. The formulation of claim 35 further comprising one or more flavors.

37. The formulation of claim 36 further comprising one or more buffering agents.

38. The formulation of claim 37 further comprising one or more preservatives.

39. The formulation of claim 37 further comprising one or more solubility enhancing agents.

40. The formulation of claim 37 further comprising one or more complexation-enhancing agents.
